SEQUENCE LISTING (8 sequences)

SEQ ID NO: 1 - protein, 24 aa, Artificial Sequence
  synthetic construct; HCV E2 fragment
INTNGSWHINSTALNCNESLNTGW

SEQ ID NO: 2 - protein, 9 aa, Artificial Sequence
  synthetic construct; HCV E2 fragment
FNSSGCPER

SEQ ID NO: 3 - protein, 11 aa, Artificial Sequence
  synthetic construct; HCV E2 fragment
ANGSGLDERPY

SEQ ID NO: 4 - protein, 22 aa, Artificial Sequence
  synthetic construct; HCV E2 fragment
SWGANDTDVFVLNNTRPPLGNW

SEQ ID NO: 5 - protein, 6 aa, Artificial Sequence
  synthetic construct; HCV E2 fragment
MNSTGF

SEQ ID NO: 6 - protein, 18 aa, Artificial Sequence
  synthetic construct; HCV E2 fragment
VCGAPPCVIGGVGNNTLL

SEQ ID NO: 7 - protein, 8 aa, Artificial Sequence
  synthetic construct; HCV E2 fragment
HYPCTINY

SEQ ID NO: 8 - protein, 7 aa, Artificial Sequence
  synthetic construct; HCV E2 fragment
LEAACNW

(19) United States
(12) Patent Application Publication
(10) Pub. No.: US 2024/0100154 A1
(43) Pub. Date: Mar. 28, 2024

(54) COMPOSITIONS AND METHODS FOR PRODUCING GLYCO-MODIFIED VIRAL ANTIGENS

(71) Applicants: UNIVERSITY OF MARYLAND, COLLEGE PARK, College Park, MD (US); DANMARKS TEKNISKE UNIVERSITET, Lyngby (DK); THE REGENTS OF THE UNIVERSITY OF CALIFORNIA, Oakland, CA (US)

(72) Inventors: Thomas R. Fuerst, Gaithersburg, MD (US); Eric A. Toth, Spring Grove, PA (US); Nathan Lewis, San Diego, CA (US); Bjorn Gunnar Voldborg, Lyngby (DK); Wan Tien Chiang, La Jolla, CA (US)

(21) Appl. No.: 18/274,741

(22) PCT Filed: Jan. 28, 2022
(86) PCT No.: PCT/US2022/014338

§ 371 Date: Jul. 28, 2023

Related U.S. Application Data
(60) Provisional application No. 63/143,410, filed on Jan. 29, 2021.

(51) Int. Cl. A61K 39/29; A61K 49/00; C07K 1/107; C07K 14/005; C12N 7/00; C12N 15/86; G01N 33/569

(52) U.S. Cl. CPC A61K 39/29; A61K 49/0004; C07K 1/1077; C07K 14/005; C12N 7/00; C12N 15/86; G01N 33/56983; A61K 2039/6087

(57) ABSTRACT

Disclosed herein are methods of producing glyco-modified viral antigens that provide a shift of the glycosylation profile of recombinant produced viral antigens (e.g. glycoproteins) towards the naturally occurring viral antigens (e.g. glycoproteins). Disclosed are methods of producing a modified viral antigen comprising expressing a viral antigen in a recombinant mammalian cell line having one or more of the endogenous genes Mgat2, Mgat4A, Mgat4B, Mgat5, St3Gal3, St3Gal4, B4galt1, B4galt2, B4galt3, B4galt4, B4galt5, B3gnt2, St3Gal6, SPPL3, and/or FUT8 inactivated and/or downregulated; and optionally a gene ST6Gall inserted. Disclosed are glyco-modified viral antigens produced by the method of using a recombinant mammalian cell line. Disclosed are methods of treating a subject in need thereof comprising administering a composition comprising a therapeutically effective amount of one or more of the glyco-modified viral antigens. Disclosed are methods of screening for an antibody specific to one or more of the disclosed glyco-modified viral antigens.

### CROSS-REFERENCE TO RELATED APPLICATIONS

This application claims the benefit of U.S. Provisional Patent Application No. 63/143,410, filed on Jan. 29, 2021, which is incorporated by reference herein in its entirety.

### STATEMENT REGARDING FEDERALLY SPONSORED RESEARCH

This invention was made with government support under Grant Number RO1AI132213 awarded by the National Institutes of Health. The government has certain rights in this invention.

### BACKGROUND

Infectious diseases pose a substantial burden on humanity and animals. One such disease is viral hepatitis from Hepatitis A, B, or C virus infections. These can trigger serious life-threatening liver disease, and are a leading cause of hepatocellular carcinoma. Globally, most deaths from hepatitis stem from hepatitis B and C. The World Health Organization (WHO) estimates that in 2015, the prevalence of HBV infection was 257 million and HCV infection was 71 million. Furthermore, it affects up to 500 million people worldwide and accounts for more than 1.6 million deaths every year. Vaccines are considered the best way to prevent infectious diseases, including viral hepatitis. However, while vaccines exist for hepatitis A and hepatitis B infections, there remains no effective vaccine available for preventing hepatitis C virus infection. There is a clear and urgent need for developing potent and effective HCV vaccines.

Major advances in the study of glycosylation in the past decades have enabled the development of recombinant vaccines using glycoengineering technologies. Evidence, however, has indicated that glycosylation can be important in vaccine development. Thus, glycoengineering can be valuable in optimizing vaccines for a variety of infectious diseases such as Covid-19 and hepatitis B.

### BRIEF SUMMARY

Disclosed herein are methods of producing glyco-modified viral antigens that provide a shift of the glycosylation profile of recombinant produced viral antigens (e.g. glycoproteins) towards the naturally occurring viral antigens (e.g. glycoproteins).

Disclosed are methods of producing a modified viral antigen comprising expressing a viral antigen in a recombinant mammalian cell line having one or more of the endogenous genes Mgat2, Mgat4A, Mgat4B, Mgat5, St3Gal3, St3Gal4, B4galt1, B4galt2, B4galt3, B4galt4, B4galt5, B3gnt2, St3Gal6, SPPL3, and/or FUT8 inactivated and/or downregulated; and optionally a ST6Gall gene.

Disclosed are glyco-modified viral antigens produced by the method of using a recombinant mammalian cell line having one or more of the endogenous genes Mgat2, Mgat4A, Mgat4B, Mgat5, St3Gal3, St3Gal4, B4galt1, B4galt2, B4galt3, B4galt4, B4galt5, B3gnt2, St3Gal6, SPPL3, and/or FUT8 inactivated and/or downregulated; and optionally a ST6Gall gene. Thus, disclosed are glyco-modified viral antigens comprising a glycan structure of one or more of the structures of Table 1.

Disclosed are methods of treating a subject in need thereof comprising administering a composition comprising a therapeutically effective amount of one or more of the glyco-modified viral antigens disclosed herein.

Disclosed are methods of treating a subject having HCV comprising administering a composition comprising a therapeutically effective amount of one or more of the glyco-modified HCV antigens disclosed herein.

Disclosed are methods of inducing an immune response in a subject in need thereof comprising administering to the subject in need thereof a composition comprising a glyco-modified viral antigen produced using one or more of the methods described herein or using one or more of the glyco-modified viral antigens described herein.

Disclosed are methods of identifying an antibody specific to one or more of the glyco-modified viral antigens described herein; comprising contacting a cell with one or more of the glyco-modified viral antigens under conditions to allow for the cell to elicit an immune response, isolating an antibody that specifically binds to the one or more of the glyco-modified viral antigens.

Disclosed are methods of generating an antibody specific to one or more of the disclosed glyco-modified viral antigens produced using the methods described herein; comprising contacting a cell with one or more of the disclosed glyco-modified viral antigens under conditions to allow for the cell to elicit an immune response, thus producing antibodies, and isolating an antibody that specifically binds to one or more of the glyco-modified viral antigens.

Disclosed are methods of screening for an antibody specific to one or more of the disclosed glyco-modified viral antigens.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B provide schematic diagrams of the E1E2 signal peptide and expression constructs. (A) (Top panel) Schematic of the E1E2 signal peptide (SP) plus polyprotein expression construct. (Bottom panel) Processing pathway for the N-terminal portion of the HCV polyprotein. Signal peptidase cleavages release E1 and E2. Signal peptide peptidase (SPP) cleaves HCV core. Repositioning of E1 and E2 transmembrane domains (TMDs) is indicated by curved arrows. Glycans attached to E1 and E2 depicted as branched structures. (B) The expression construct used for this disclosure contains just the E2 domain, engineered to be released and secreted.

FIG. 2 shows the glycosylation for HEK293-sE2 and Sf9-sE2, and the localization of nAb binding locations. Schematic representation of HCV sE2 chymotryptic glycopeptides depicted with the most abundant glycans observed during LC-MS analysis of HEK293 (top glycoform) and Sf9 (bottom glycoform) sE2 proteins. FIG. 2 shows the glycans (N1-N11) expressed on the sE2 glycoproteins produced in the HEK293 cell line and the Sf9 cell line. The four domains (Domain A, B, D, and E) on sE2 protein were depicted that were bound by the four tested nAbs (CBH-4D, AR3A, HC84.26, and HCV1).

FIGS. 3A-3C is an example of a process of generating glycoengineered subunit vaccines. (FIG. 3A) A schematic view of subunit vaccine proteins being produced in the CHO cells. For example, the engineered envelope sE2 glycoprotein of HCV can be produced by CHO cells. (FIG. 3B) The sE2 protein glycoforms can be produced by different glycoengineered CHO cell lines to create different glycoforms. nAb assay can be applied to assess the binding strength (Kd and Bmax) of antibodies on each sE2 protein glycoform (see Methods for details). (FIG. 3C) The binding strength relationships between antibodies and specific glycan epitopes can be learned using a machine learning pipeline (ML-pipeline). The most effective glycan epitope will be selected for designing the glycan-vaccine for protecting people against virus (HCV) infection.

FIGS. 4A and 4B show a GlycoEpitope Positioning System (GEPS). (FIG. 4A) A schematic view of the GEPS system that can be used to identify the best glycan epitope for interacting with monoclonal antibodies (or any other biological response). There are four major functionalities in the GEPS system: 1) Positioning—positioning the best glycan epitope for nAb interactions, 2) Annotation—annotating the glycoprofile of glycan-vaccine candidates with different levels of glycan features (glycan structure, glycan substructure, and epitope), 3) Analyzing—quantifying the binding strength between antibodies and the annotated glycan features, and 4) Prediction—the built model can be further used in predicting the glycan features for designing glycoprotein vaccines. (FIG. 4B) Shows examples of 13 well-known glycan epitopes of the N-linked glycan.

FIGS. 5A-5D shows examples of glycoforms of liver-secreted proteins differ substantially from glycoforms produced recombinantly. (FIG. 5A) In vivo, the major liver secreted proteins mostly have more simple afucosylated biantennary glycans. (FIG. 5B) However, expression of alpha-1 antitrypsin from a stable CHO-S clone shows substantially higher heterogeneity and is almost completely missing the dominant glycans seen on the native protein secreted from the human liver. (FIG. 5C) A screen of transiently expressed alpha-1 antitrypsin in the glycoengineered strains, the in vivo human glycans were essentially missing, but one clone was found wherein it dominated. (FIG. 5D) sE2 expressed in CHO-S cells only missing one glycosyltransferase (B3gnt2) showed the high heterogeneity of recombinant sE2 glycosylation, which substantially differs from glycosylation on endogenous liver-secreted proteins.

FIGS. 6A and 6B show examples of the glycoprofiles of recombinant sE2 produced in WT and glycoengineered CHO cells. (FIG. 6A) Thirteen cell lines from our panel were used to secrete sE2 glycoproteins, each with a different dominant glycoform. (FIG. 6B) Heatmap of a recombinant sE2 proteins with diverse N-linked glycans (columns), each produced in a different geCHO cell line (rows). The sE2 protein produced in strains #10 (monomer and aggregate samples, labeled .m and .a, respectively) contains a set of large glycans (m/z>2500).

FIGS. 7A-7D show examples of the geCHO cells expressing sE2 and their subsequent nAb assay. Patient-derived anti-E2 nAbs were tested via ELISA to assess their binding strength (Kd and Bmax; See also Table 2) on each glycoengineered sE2 protein. Correlation analysis of nAb assay for the binding strength (Kd) of antibodies elicited by glycoengineered sE2 proteins against HCV without sE2 strain #10 (FIG. 7A) and with sE2 strain #10 (FIG. 7B), and for the Bmax without sE2 strain #10 (FIG. 7C) and with sE2 strain #10 (FIG. 7D). The correlation analysis conducted here is the Spearman's rank correlation, and the number shown here is the Spearman's rank correlation coefficient. The notations of significance are: *P<0.05; **P<0.01; ***P<0.001.

FIGS. 8A and 8B provide an example of a correlation analysis of glycan abundance and nAb binding strength (Kd). (FIG. 8A) The correlation analysis conducted here is the Spearman's rank correlation, and the number shown here is the Spearman's rank correlation coefficient. The rectangle highlights the two glycans (m/z=1395.5 and 1436.5) show significant correlations with nAb binding strength (Kd). (FIG. 8B) Scatter plots of the correlations between the indicated nAb and the two glycans m/z=1395.5 (up panels) and 1436.5 (bottom panels). From this it is clear that improved nAb binding is seen with fewer branches on the glycans but with full galactosylation of the branch, as seen on the native liver glycans. The notations of significance are: *P<0.05; **P<0.01; ***P<0.001. The lower KD value denotes the higher affinity of the antibody. The vaccine design prefers the glycan that has one branch, asialylated and afucosylated (m/z=1395). The vaccine design would like to prevent agalactosylation (m/z=1436).

FIGS. 9A and 9B show a correlation analysis of glycan epitope abundance and nAb binding strength (Kd). (FIG. 9A) The correlation analysis conducted here is the Spearman's rank correlation, and the number shown here is the Spearman's rank correlation coefficient. The rectangles highlight the two glycan epitopes, exposed GlcNAc (recognized by Nictaba lectin) and biantennary GlcNAc (recognized by WGA lectin), show significant correlations with nAb binding strength (Kd). (FIG. 9B) Scatterplots showing the correlations between the indicated nAb and the two identified glycan epitopes indicated by their bound lectins-Nictaba (up panels) and WGA (bottom panels). From these results it is clear that improved nAb binding is seen with full galactosylation of branches and sialylation of branches, as seen on the native liver glycans. The notations of significance are: *P<0.05; **P<0.01; ***P<0.001. The lower KD value denotes the higher affinity of the antibody. The vaccine design would like to prevent agalactosylation (exposed GlcNAc epitope: Nictaba or WGA).

FIGS. 10A-10D show example multiple linear regression modeling of the associations between glycan epitope abundance and nAb binding strength (Kd). The scatter plots present the multiple linear regression models for the associations between glycan epitope abundance and nAb binding strength (Kd) of the four studied antibodies: CBH-4D (FIG. 10A), AR3A (FIG. 10B), HC84.26 (FIG. 10C), and HCV1 (FIG. 10D). Note that, ‘R²’ denotes the goodness-of-fit (i.e., the % of variance in the Kd can be explained using the indicated model) for linear regression model applied to the training dataset, and ‘CV_R²’ denotes goodness-of-fit for the models applied to the leave-one-out cross validation testing dataset.

FIGS. 11A-11D show examples of an assessment of the importance of glycan epitopes in the multiple linear regression models. The barplots show the importance of glycan epitopes in the multiple linear regression models for the four studied antibodies: CBH-4D (FIG. 11A), AR3A (FIG. 11B), HC84.26 (FIG. 11C), and HCV1 (FIG. 11D). The importance of glycan epitopes was quantified by the Correlation-Adjusted coRelation (CAR) scores.³⁶ The CAR score is a variance decomposition method that is defined as the marginal correlations adjusted for correlation among explanatory variables.

FIGS. 12A-12C show examples of learning the associations between glycan epitope abundance and nAb binding strength (Kd) using neural networks. (FIG. 12A) A schematic view of the framework of the neural network-based method for predicting the Kd for any given glycoprofile: the lectin/glycan profile (input), the predicted nAb binding strength (Kd) (output), and the neural network with two hidden layers (grey shaded) and neurons. (FIG. 12B, FIG. 12C) The boxplots of performance (R; correlation coefficient between experimental and prediction) for the nAb binding strength (Kd) prediction from their corresponding glycan/epitope profiles using different neural network structures (number of layers and neurons). Each box represents the performance of leave-one-out cross validation of 100 random neural networks with the indicated topology.

FIGS. 13A and 13B show neural network models for predicting nAb binding strength (Kd). The best neural network models using glycan profile (FIG. 13A) or epitope profile (FIG. 13B) as predictors. (left) The scatter plot of predicted nAb binding strength (Kd) versus experimental nAb binding strength (Kd). (right) The relative lectin importance of the best performance neural network.

FIGS. 14A and 14B show a glycan epitope effect analysis. (FIG. 14A) The K_{d} profile plot of changing each epitope while holding others constant at their specified quantiles (0% (min), 25%, 50%, 75%, or 100% (max)). The red rectangles highlight the epitopes that have the maximum effect for the indicated nAbs. (FIG. 14B) Effect plots of changing glycan epitope abundance while holding others constant at their specified quantiles. These emphasize the benefit of having fewer branches but more galactosylation of the branches.

FIG. 15 shows in-silico clone screening results. 10⁵ different combinations of epitope profiles were sampled using the Latin Hypercube Sampling (LHS) algorithm³⁷. These samples were further used with the best NNₑₚᵢₜₒₚₑ model to predict their nAb binding strength (K_{d}). The heatmap shows the epitope profiles for the 10⁵ samples, which are sorted by the ‘Total score (K_{d})’. The right panels were the predicted K_{d} values of indicated nAb, and the ‘Total score (K_{d})’ is the summation of 4 K_{d} values of each sample. The epitope abundances of the top 5 samples were presented in the top panel by bar plots.

FIG. 16 shows a distribution of the In-silico clone screening results. The histogram shows the distribution of the NNₑₚᵢₜₒₚₑ model predicted total score (K_{d}) for the 10⁵ LHS samples. The blue dashed lines indicate the experimentally measured total score (K_{d}) of the six geCHO profiles (#1, 2, 4, 5, 6, 7). The red dashed line indicates the best predicted total score (K_{d}) of the total 10⁵ LHS samples. Note that, here the indicated six geCHO samples were shown. The experimentally measured total score (K_{d}) for the WT(CHO) (5.76), WT(HEK) (2.39), and geCHO(#10) (6.84) were not shown, because their total score (K_{d}) is much higher than 2.

FIGS. 17A-B shows N-glycomics of CHO cells expressing sE2 #1. Glycoprofiling of monomer (FIG. 17A) and aggregate (FIG. 17B) states of sE2 expressed in CHO cells (knockout of st3gal3/4/6, b3gnt2, and sppl3 genes involved in N-glycosylation). The peaks represent LC-MS spectra of peptide-N-glycosidase-F-released permethylated N-glycans. The y-axis presents the relative abundances of indicated time (min).

FIGS. 18 A-B shows N-glycomics of CHO cells expressing sE2 #2. Glycoprofiling of monomer (FIG. 18A) and aggregate (FIG. 18B) states of sE2 expressed in CHO cells (knockout of st3gal3/4/6, b3gnt2, fut8, and mgat2 genes involved in N-glycosylation). The notations are the same as those defined in FIGS. 17A-B.

FIGS. 19A-B shows N-glycomics of CHO cells expressing sE2 #4. Glycoprofiling of monomer (FIG. 19A) and aggregate (FIG. 19B) states of sE2 expressed in CHO cells (knock in of human St6gal1 gene and knockout of st3gal3/4/6, b3gnt2, mgat2, fut8, and sppl3 genes involved in N-glycosylation). The notations are the same as those defined in FIGS. 17A-B.

FIGS. 20A-B shows N-glycomics of CHO cells expressing sE2 #5. Glycoprofiling of monomer (FIG. 20A) and aggregate (FIG. 20B) states of sE2 expressed in CHO cells (knockout of mgat4a/4b/5, b4galt1/2/3, and b3gnt2 genes involved in N-glycosylation). The notations are the same as those defined in FIGS. 17A-B.

FIGS. 21A-C shows N-glycomics of CHO cells expressing sE2 #6. Glycoprofiling of monomer (FIG. 21A) and aggregate (FIG. 21B, FIG. 21C) states of sE2 expressed in CHO cells (knockout of mgat4a/4b/5, st3gal3/4/6, b3gnt2, fut8, and sppl3 genes involved in N-glycosylation). The notations are the same as those defined in FIGS. 17A-B.

FIGS. 22A-B shows N-glycomics of CHO cells expressing sE2 #7. Glycoprofiling of monomer (FIG. 22A) and aggregate (FIG. 22B) states of sE2 expressed in CHO cells (knock in of human St6gal1 gene and knockout of mgat4a/4b/5, st3gal3/4/6, b3gnt2, fut8, and sppl3 genes involved in N-glycosylation). The notations are the same as those defined in FIGS. 17A-B.

FIGS. 23A-B shows N-glycomics of CHO cells expressing sE2 #10. Glycoprofiling of monomer (FIG. 23A) and aggregate (FIG. 23B) states of sE2 expressed in CHO cells (knockout of b3gnt2 and sppl3 genes involved in N-glycosylation). The notations are the same as those defined in FIGS. 17A-B.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a viral antigen” includes a plurality of such viral antigens, reference to “the viral antigen” is a reference to one or more viral antigens and equivalents thereof known to those skilled in the art, and so forth.

As used herein, the term “subject” or “patient” can be used interchangeably and refer to any organism to which a protein (e.g. viral antigen) or composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, “glyco-modified viral antigen” refers to a viral antigen in which the glycosylation pattern has been altered. In some aspects, “glyco-modified viral antigen” refers to a viral antigen in which the glycosylation pattern has been altered from that produced in vitro in a mammalian cell line to that of a mammalian cell line engineered to glycosylate a viral antigen to comprise a specific glycoprofile. For example, a glyco-modified HCV E2 can be a recombinantly produced to have a predetermined or specific glycoprofile.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Producing Glyco-Modified Viral Antigens

Glyco-analysis of viral antigens revealed that a naturally occurring dominant viral antigen is glycosylated with a fully sialylated bi-antennary structure without core fucosylation. Glyco-analysis of proteins secreted from the liver show they are predominantly glycosylated with a fully sialylated bi-antennary structure without core fucosylation. In contrast, CHO-produced liver proteins, and viral antigens can contain many different structures with a partially and fully sialylated bi-, tri-, and tetra-antennary structures with core fucosylation as the most dominant species.

Disclosed herein are methods of producing glyco-modified viral antigens that provide a particular glycosylation profile of recombinant produced viral antigens (e.g. glycoproteins). In some aspects, the glyco-modified viral antigen is a viral antigen in which the glycosylation pattern has been altered from that produced in vitro in a mammalian cell line to that of a mammalian cell line engineered to glycosylate a viral antigen to comprise a specific glycoprofile. In some aspects, disclosed herein are methods of producing glyco-modified viral antigens that provide a particular glycosylation profile of recombinant produced viral antigens similar to the naturally occurring viral antigens (e.g. glycoproteins). These methods can include knocking out or in any other way downregulating a selected a set of glycosylating enzymes. This can result in a 6, 7, 8, 9, 10 or 8-9 double knock out clone in which, glycoproteins, such as viral antigens (e.g. HCV E2) can be expressed.

Disclosed are methods of producing a glyco-modified viral antigen comprising expressing a viral antigen in a recombinant mammalian cell line, wherein the recombinant mammalian cell line is designed to provide a glycosylation pattern on viral antigens that more closely resembles that of the naturally occurring viral antigen. The recombinant mammalian cell lines that can be used in the disclosed methods are described herein. For example, disclosed are methods of producing a glyco-modified viral antigen comprising expressing a viral antigen in a recombinant mammalian cell line having one or more of the endogenous genes Mgat2, Mgat4A, Mgat4B, Mgat5, St3Gal3, St3Gal4, B4galt1, B4galt2, B4galt3, B4galt4, B4galt5, B3gnt2; and St3Gal6, SPPL3, and/or FUT8 inactivated and/or downregulated. The recombinant mammalian cell line can optionally comprise a ST6Gall gene. In some aspects, the recombinant mammalian cell line can be one or more of the cell lines described in PCT Application Publication WO 2019/105770, which is hereby incorporated by reference in its entirety for its teaching of recombinant mammalian cell lines.

1. Recombinant Mammalian Cell Line

In some aspects, the recombinant mammalian cells used to produce a glyco-modified viral antigen can comprise one or more of the endogenous genes Mgat2, Mgat4A, Mgat4B, Mgat5, St3Gal3, St3Gal4, B4galt1, B4galt2, B4galt3, B4galt4, B4galt5, B3gnt2; St3Gal6, SPPL3, and/or FUT8 inactivated and/or downregulated; and optionally a ST6Gall gene. The inactivation and/or downregulation can be a result of any gene manipulation techniques (e.g., knockout, knock in, knock down and/or any regulation of the interested gene), or through chemical or drug treatment.

In some aspects, one or more of the following modifications can be made to the recombinant mammalian cells used to produce a glyco-modified viral antigen: 1) inactivation and/or downregulation of a series of enzymes Mgat4A, Mgat4B, and Mgat5 that facilitate a decrease in branching; 2) inactivation and/or downregulation of a series of enzymes St3Gal3, St3Gal4, and St3Gal6 that facilitate the removal of CHO specific alpha-2, 3-sialylation; 3) inactivation and/or downregulation of the enzyme SPPL3 that facilitate to increase glycosyltransferases half-life in the Golgi; 4) inactivation and/or downregulation of the enzyme FUT8 that facilitate the removal of core-fucosylation; 5) inactivation and/or downregulation of the enzyme B3GNT2 that can remove elongated antennas; 6) inactivation and/or downregulation of the enzyme GLUL that can boost cell growth, and may be used for selection; and 7) insertion of a gene encoding Beta-galactoside alpha-2, 6-sialyltransferase 1 (St6gall), which gene directs a human type branching of terminal sialic acids.

In some aspects, one or more of the disclosed modifications can result in a shift in the glycosylation profile to the predominant bi-antennary form similar to a naturally occurring virus from that of recombinant produced viral antigens, such as HCV E1 produced in CHO cells.

In some aspects, one or more of the recombinant mammalian cells described herein can be modified by insertion of a gene expressing an exogenous human glycoprotein of interest, such as a viral antigen, wherein the viral antigen is from Herpesvirus, Poxvirus, Hepadnavirus, Asfarviridae, Flavivirus, Alphavirus, Togavirus, Coronavirus, Hepatitis D, Orthomyxovirus, Paramyxovirus, Rhabdovirus, Bunyavirus, or Filovirus.

In some embodiments the mammalian cells used in the disclosed methods can be, but are not limited to, Chinese Hamster Ovarian (CHO) cells, such as CHO-Kl; Baby Hamster Kidney (BHK) cell; COS cell; HEK293; HeLa; NSO; SP2/0; YB2/0; HUVEC; HKB; PER-C6; or derivatives of any of these cells.

2. Viral Antigen

In some aspects, a glyco-modified viral antigen is a glyco-modified viral antigen. Thus, in some aspects, a glyco-modified viral antigen is a glycoprotein derived from a virus. Examples of glycoproteins derived from a virus can be, but are not limited to, Hemagglutinin and Neuraminidase (influenza); spike glycoprotein (SARS-Cov); E1 and E2 (hepatitis C virus); gp120, gp160, gp41 (HIV-1), spike protein Gp1-Gp2 (Zaire Ebola virus), E (Dengue virus), E1 and E2 (Chikungunya virus).

In some aspects, the viral antigen is a viral antigen from an enveloped virus. In some aspects, the enveloped virus is a DNA virus, RNA virus or a retrovirus. Examples of a DNA virus are, but are not limited to, a Herpesvirus, Poxvirus, Hepadnavirus, and Asfarviridae. Examples of a DNA virus are, but are not limited to, Flavivirus, Alphavirus, Togavirus, Coronavirus, Hepatitis D, Orthomyxovirus, Paramyxovirus, Rhabdovirus, Bunyavirus, and Filovirus.

In some aspects, a glyco-modified viral antigen has increased antigenicity compared to the viral antigen not glyco-modified. In some aspects, the glyco-modified antigen can be generated with modifications to the natural antigen for increasing the antigenicity. In some aspects, a glyco-modified viral antigen has increased or equal antigenicity compared to the natural viral antigen.

In some aspects, the glyco-modified viral antigen comprises one or more of the following glycan structures: a primary n-glycan structure that is a fully sialylated bi-antennary structure without core fucosylation, such as with more than 80%, such as 82%, such as 84%, such as 86%, such as 88%, such as 90% of the glyco-modified viral antigen produced being in with a fully sialylated bi-antennary structure without core fucosylation; a glycan structure according to the structure A2G2S2 with the following pictorial representation:

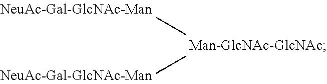

a glycan structure according to the structure:

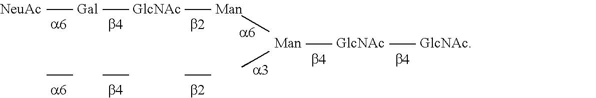

In some aspects, the glyco-modified viral antigen comprises one or more of the following glycan structures: mono-antennary, no sialic acids (FA1G1); mono-antennary non-fucosylated, no sialic acids (A1G1); or mono-antennary non-fucosylated (FA1G1S1).

In some aspects, the glyco-modified viral antigen comprises one or more of the glycan structures described in Table 1.

TABLE 1
Examples of glycan structures

| Glycoform | |
|---|---|
| (Oxford | |
| Notation) | description |
| A2 | Biantennary non-fucosylated, no galactoses, no sialic |
| | acids |
| FA2 | Biantennary no galactoses, no sialic acids |
| A2G2 | Biantennary non-fucosylated, no sialic acids |
| FA2G2 | Biantennary no sialic acids |
| A2G1 | Biantennary non-fucosylated, one galactose, no sialic |
| | acids |
| FA2G1 | Biantennary one galactose, no sialic acids |
| A2G2S1(3) | Biantennary non-fucosylated, one α2,3 linked sialic acid |
| FA2G2S1(3) | Biantennary one α2,3 linked sialic acid |
| A2G2S1(6) | Biantennary non-fucosylated, one α2,6 linked sialic acid |
| FA2G2S1(6) | Biantennary one α2,6 linked sialic acid |
| A2G2S2(3) | Biantennary non-fucosylated α2,3 linked sialic acids |
| FA2G2S2(3) | Biantennary α2,3 linked sialic acids |
| A2G2S2(6) | Biantennary non-fucosylated α2,6 linked sialic acids |
| FA2G2S2(6) | Biantennary α2,6 linked sialic acids |

In some aspects, the glyco-modified viral antigen has a glycoprofile that is similar to that of the host tissue wherein the virus replicates. For example, if the glyco-modified viral antigen is a HCV glycoprotein, then the glycoprofile can match that of the HCV glycoprotein in the liver since HCV replicates in the liver.

In some aspects, the viral antigen is a HCV antigen, thus the glyco-modified viral antigen can be a glyco-modified HCV antigen. In some aspects, the glycosylation approximates the glycosylation from native liver-secreted proteins. In some aspects, the dominant N-linked glycans are biantennary sialylated, afucosylated glycans or galactosylated paucimannose or asialylated. In some aspects, the dominant N-linked glycans are devoid of agalactosylated branches.

In some aspects, the viral antigen is a modified HCV antigen. In some aspects, the viral antigen is a modified hepatitis C virus (HCV) E2 glycoprotein comprising an antigenic domain D, wherein the modified hepatitis C virus (HCV) E2 glycoprotein comprises one or more amino acid alterations in the antigenic domain D. In some aspects, the “modified HCV antigen” is a modification other than the glyco-modification. For example, a modified HCV E2 can comprise one or more amino acid alterations.

In some aspects, the viral antigen is a modified membrane-bound hepatitis C virus (HCV) E1E2 glycoprotein comprising an HCV E1 glycoprotein and a modified HCV E2 glycoprotein, wherein the modified HCV E2 glycoprotein comprises an antigenic domain D, wherein the modified HCV E2 glycoproteins comprise one or more amino acid alterations in the antigenic domain D.

In some aspects, the viral antigen is a modified HCV E1E2 glycoprotein comprising: a HCV E1 polypeptide, a first scaffold element, a HCV E2 polypeptide, and a second scaffold element, wherein the HCV E1 polypeptide does not comprise a transmembrane domain, and wherein the HCV E2 polypeptide does not comprise a transmembrane domain.

The HCV E2 and HCV E1E2 polypeptides used in the disclosed methods can be one or more of those HCV polypeptides described in PCT Application PCT/US2021/059171, incorporated by reference in its entirety.

In some aspects, an amino acid alteration in the antigenic domain D of HCV E2 can be an amino acid substitution, deletion, or addition.

### C. Glyco-Modified Viral Antigens

Disclosed are glyco-modified viral antigens produced by one or more of the methods disclosed throughout. Thus, disclosed are recombinantly produced glyco-modified viral antigens.

In some aspects, a glyco-modified viral antigen can be Haemaglutinin and Neuraminidase (influenza); spike glycoprotein (SARS-CoV); E1 and E2 (hepatitis C virus); gp120, gp160, gp41 (HIV-1), Gp1-Gp2 (Ebola virus), E (Dengue virus), E1 and E2 (Chikungunya virus), F and G (respiratory syncytial virus), gB, gD, gH, gL (herpes simplex virus), among others.

Disclosed is a glyco-modified HCV E2 glycoprotein. In some aspects, the HCV E2 glycoprotein is a modified HCV E2 glycoprotein comprising an antigenic domain D, wherein the modified HCV E2 glycoprotein comprises one or more amino acid alterations in the antigenic domain D and further comprising one or more of the glycan structures of Table 1, thus making the modified HCV E2 also glyco-modified. For example, disclosed is a glyco-modified, modified HCV E2 glycoprotein comprising an antigenic domain D, wherein the modified HCV E2 glycoprotein comprises one or more amino acid alterations in the antigenic domain D and further comprising the A2G2S2(6) (biantennary non-fucosylated α2,6 linked sialic acids) glycan structure (thus making the modified HCV E2 a glyco-modified, modified HCV E2).

### D. Methods of Treating

Disclosed are methods of treating a subject in need thereof comprising administering a composition comprising a therapeutically effective amount of one or more of the glyco-modified viral antigens described herein. For example, the glyco-modified viral antigen can be any glyco-modified viral antigen having a glycan structure of one or more of those described in Table 1. In some aspects, the glyco-modified viral antigen can be one produced using the methods described herein.

In some aspects, a subject in need thereof is a subject having a viral infection or at risk of being infected with a virus, wherein the glyco-modified viral antigen is a viral antigen associated with the viral infection. For example, if a subject has HCV then the glyco-modified viral antigen can be a glyco-modified HCV, such as a glyco-modified E2.

Disclosed are methods of treating a subject having HCV comprising administering to the subject a composition comprising one or more of the glyco-modified viral antigens. In some aspects, the glyco-modified viral antigen is a HCV glycoprotein or a modified HCV glycoprotein. In some aspects, the glyco-modified viral antigen has one or more glycan structures described in Table 1.

In some aspects, the disclosed methods of treating occur by the subject generating an immune response, such as antibodies, to the glyco-modified viral antigen, wherein the immune response fights a viral infection or prevents a viral infection. For example, neutralizing antibodies (nAbs) can be produced upon administering a composition comprising a therapeutically effective amount of one or more of the glyco-modified viral antigens, wherein the nAbs can prevent or reduce viral infection.

In some aspects, the immune response generated to one or more of the glyco-modified viral antigens is at least 1×, 2×, 3×4×, 5×, 6×, 7×, 8×, 9×, 10×, 20×, 30×, 40×, 50×, 60×, 70×, 80×, 90× or 100× greater than the immune response generated to the same viral antigen produced recombinantly but that is not glyco-modified.

### E. Methods Related to Immune Responses

Disclosed are methods of increasing HCV E2 antigenicity in a subject in need thereof comprising administering a composition comprising a glyco-modified HCV E2 produced using one or more of the methods described herein. For example, the glyco-modified HCV E2 can be produced using any of the disclosed recombinant mammalian cell lines and can comprise one or more of the glycan structures described in Table 1.

In some aspects, a subject in need thereof is a subject having a HCV infection or at risk of being infected with HCV.

In some aspects, antigenicity is increased due to the glycan modifications made to the viral antigen.

Disclosed are methods of inducing an immune response in a subject in need thereof comprising administering to the subject in need thereof a composition comprising a glyco-modified viral antigen produced using one or more of the methods described herein or a composition comprising a glyco-modified viral antigen described herein. For example, the glyco-modified viral antigen can be produced using any of the disclosed recombinant mammalian cell lines and can comprise one or more of the glycan structures described in Table 1.

In some aspects, the glyco-modified viral antigen can be any viral glycoprotein that has been recombinantly modified with a glycoprotein pattern similar to that of the naturally occurring viral glycoprotein.

In some aspects, a subject in need thereof is a subject having a viral infection or at risk of being infected with a virus, wherein the glyco-modified viral antigen is a viral antigen associated with the viral infection. For example, if a subject has HCV then the glyco-modified viral antigen would be a modified HCV glycoprotein, such as E2.

In some aspects, the immune response can be an antibody response and/or a T cell response. In some aspects, the nature of the viral antigen is responsible for activating a T cell and/or B cell response.

Disclosed are methods of identifying an antibody specific to one or more of the glyco-modified viral antigens described herein; comprising contacting a cell with one or more of the glyco-modified viral antigens under conditions to allow for the cell to elicit an immune response, isolating an antibody that specifically binds to the one or more of the glyco-modified viral antigens.

Disclosed are methods of generating an antibody specific to one or more of the disclosed glyco-modified viral antigens produced using the methods described herein; comprising contacting a cell with one or more of the disclosed glyco-modified viral antigens under conditions to allow for the cell to elicit an immune response, thus producing antibodies, and isolating an antibody that specifically binds to one or more of the glyco-modified viral antigens.

In some aspects, the methods of identifying or generating an antibody can be carried out in a subject. In some aspects, the method can be carried out in vitro.

In some aspects, the cell being contacted with the glyco-modified viral antigen is a B cell.

In some aspects, standard approaches for cloning of monoclonal antibodies can be used. In some aspects, ex vivo expansion of cells that have been contacted by the glyco-modified viral antigen can be performed. In some aspects, use of single cell cloning methodologies can be performed.

In some aspects, the methods include differential screening of cells contacted with the glyco-modified viral antigen versus the same viral antigen but produced using standard expression technologies (not the recombinant mammalian cells developed to glyco-modify proteins). In some aspects, the antibodies produced in response to the two different viral antigens can be compared and the glycan-specific monoclonal antibodies unique to the glyco-modified viral antigen can be isolated. This would allow for isolation of glycan-specific monoclonal antibodies that antigens made using standard techniques would fail to produce. In some aspects, the glycosylation pattern on the glyco-modified viral antigens described herein can provide different antibodies compared to viral antigens produced in standard mammalian cell lines that have not been altered to provide specific glycosylation.

Disclosed are methods of screening for an antibody specific to one or more of the disclosed glyco-modified viral antigens comprising: obtaining, or having obtained, an antibody from a subject; contacting the antibody with one or more of the disclosed glyco-modified viral antigens; assessing binding strength (Kd) of the antibody to the one or more of the glyco-modified viral antigens; correlating the abundance of the one or more of the glyco-modified viral antigens and binding strength (Kd) of the antibody; wherein an improved binding strength (Kd) relative to an antibody contacted with a non-glyco-modified viral antigen is an antibody specific to one or more of the disclosed glyco-modified viral antigens. In some aspects, the binding strength difference can vary from antigen to antigen. In some aspects, there is at least a two-fold difference in binding strength between an antibody specific to one or more of the disclosed glyco-modified viral antigens and an antibody contacted with a non-glyco-modified viral antigen.

Disclosed are methods of screening for an antibody specific to one or more of the disclosed glyco-modified viral antigens comprising contacting a cell with one or more of the glyco-modified viral antigens under conditions to allow for the cell to elicit an immune response, identifying and isolating an antibody that specifically binds to the one or more of the glyco-modified viral antigens.

In some aspects, the method is carried out in a subject.

In some aspects, the methods of screening can comprise one or more aspects of the methods of identifying or generating antibodies described herein.

In some aspects, antibodies can be obtained from a subject and screened to determine which glycan structure they are most reactive with. This would allow one to determine that subjects infected with a particular virus produce a certain antibody specificity which would allow one to determine one or more of the predominant epitopes on a virus.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing one or more glyco-modified viral antigens described herein. In some aspects, the kit comprises one or more of the cell lines described here. The kits also can contain a nucleic acid sequence that encodes a viral antigen so that upon expression within one of the recombinant cell lines, the viral antigen becomes a glyco-modified viral antigen.

### EXAMPLES

### A. Example 1

1. Results

i. Glycoengineered CHO Systems can Express sE2 Protein Isoform-Glycoform at Higher Homogeneity

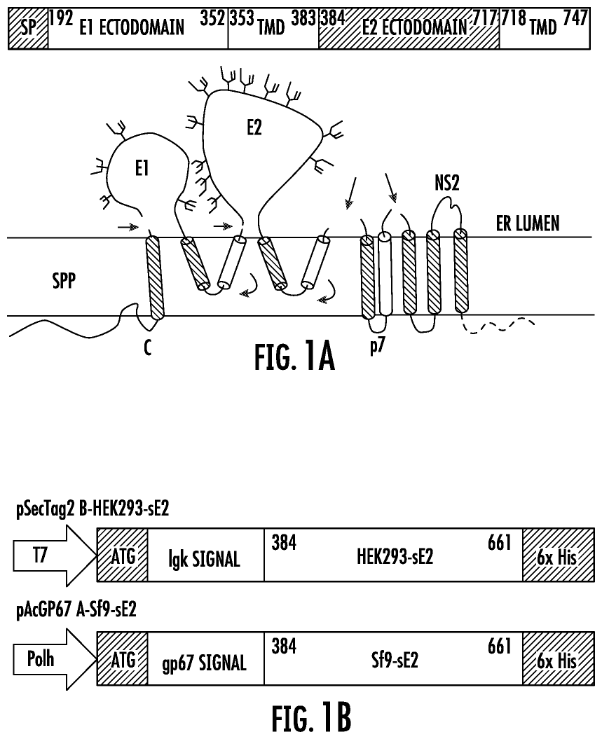

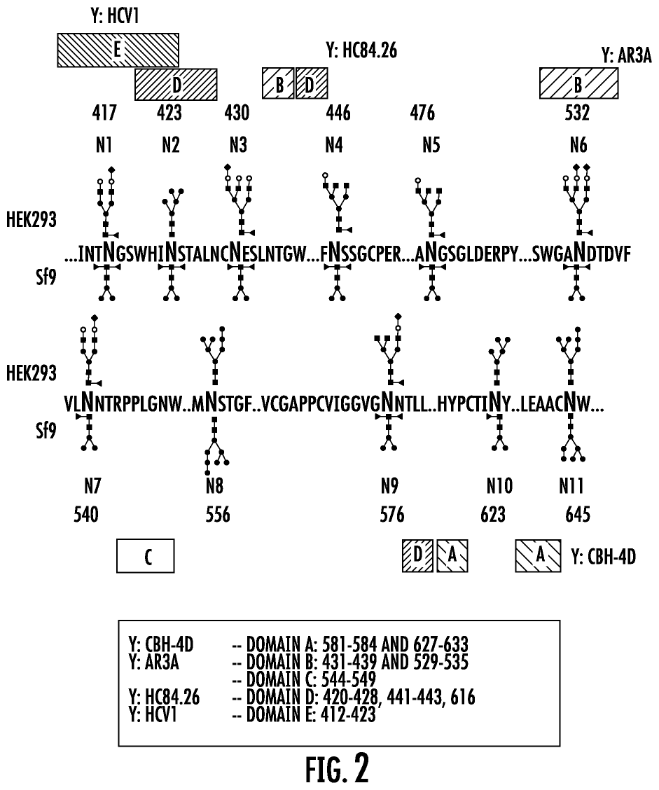

In developing a prophylactic vaccine for HCV, the choice of antigen and mode of production will be critical for its success. During the past three decades, a wealth of research provides valuable knowledge in understanding the molecular mechanisms of HCV that causes an infection predominantly in the liver cells. HCV is an enveloped virus. The envelope of HCV contains two glycoproteins, E1 and E2, which are membrane-anchored via their C-terminal helices (FIGS. 1A and B) and form a trimer of heterodimers on the surface of the virus. E1 and E2 glycoproteins are essential to help the virus bind to receptors on host cells. Glycosylation of E1 and E2 is critical to HCV entry. Recent studies demonstrate that E1/E2 glycoproteins are prime targets for a subunit vaccine that would elicit neutralizing antibody (nAb) responses. Effective vaccine candidates can mimic the native domain structures and glycosylation, or involve simple glycans to improve nAb recognition of relevant protein epitopes. One aspect of antigen production that has received little attention is the content and uniformity of the glycans on the antigens that mimic the native virion. In particular, the glycan structures in the recombinant antigen should ideally reflect those present during a viral infection of the primary host. However, when recombinant HCV secreted E2 glycoprotein ectodomain (sE2) is produced in systems like insect cells or HEK 293 cells, the antigen displays glycan structures that are both diverse and unlikely to reflect those produced by the glycosylation machinery in the liver (FIGS. 1B and 2). Likewise, similar glycan structures are expected to be seen in the E1/E2 heterodimer expressed in HEK 293 or CHO cells. Disclosed herein are HCV subunit vaccine candidates wherein the glycosylation patterns were modulated to mimic the native virion so that new light could be shed on glycosylation as a tool for rational vaccine design.

Major advances in the study of glycosylation in the past decades have enabled the development of recombinant vaccines using glycoengineering technologies. Evidence, however, has suggested that glycosylation may be important in vaccine development. Thus, glycoengineering may be valuable in optimizing vaccines for a variety of infectious diseases such as Covid-19 and hepatitis B. Here it is demonstrated that glycoengineering of a subunit vaccine candidate for HCV can 1) obtain glycosylation that mimics the host tissue glycosylation to obtain a more potent subunit vaccine, 2) identify optimal glycoforms of a subunit vaccine, and 3) identify the glycan features that are most strongly associated with a potent subunit vaccine candidate.

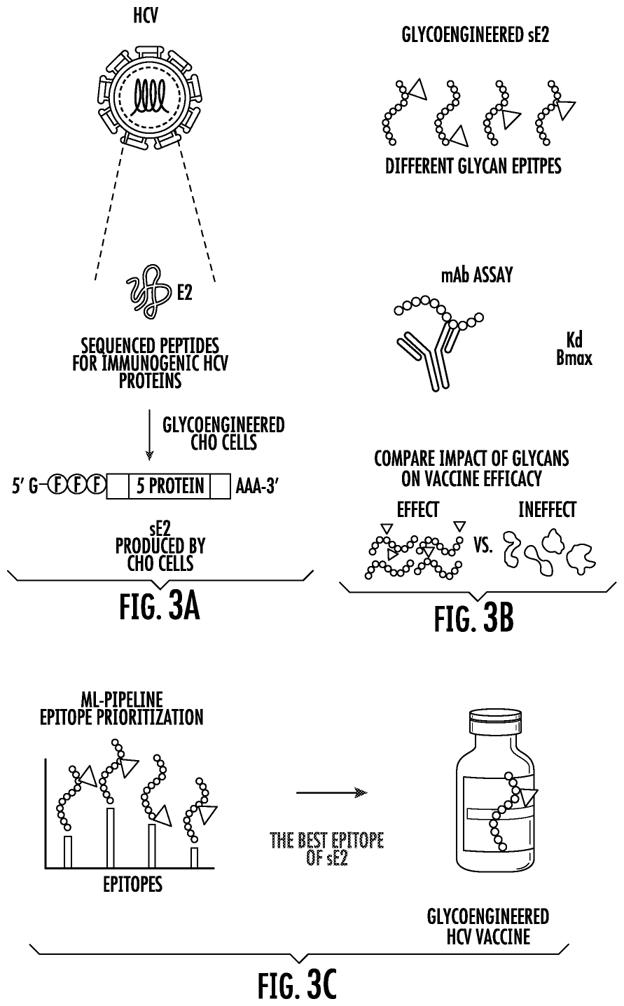

Methods to enhance envelope proteins such as E2 or E1/E2 (FIGS. 1 and 2) as subunit vaccine candidates are presented wherein glycosylation is optimized and controlled to obtain a more effective HCV vaccine. In this process, specific glycans and glycan epitopes associated with enhanced nAb binding are presented in order to design potent immunogens, i.e., antigens formulated with adjuvants, in the development of more effective vaccines. In this, HCV-E2 recombinant protein (termed as ‘sE2’) is produced in both wild-type and glycoengineered CHO (geCHO) cells to assess the impact of glycan content on antigenicity (FIG. 3A). In addition, a useful method is presented to identify glycans or glycan epitopes on the vaccine candidate protein whose abundance determines optimal vaccine efficacy (i.e., optimal binding with the assayed monoclonal antibodies) (FIG. 3B). The binding strength relationships between antibodies and glycan epitope can be learned in the machine learning pipeline (ML-pipeline). Glycan epitopes can be further selected in designing the glycan-vaccine for protecting people against virus (HCV) infection (FIG. 3C).

The recombinant secreted E2 glycoprotein often expresses a diverse set of glycoforms with subtle differences in the glycan structures decorating the protein. This heterogeneity leads to two problems. First, when glycosylation impacts function, as shown for recombinant E2, heterogeneity dilutes the efficacy of the protein product. Second, heterogeneity results in more glycans whose abundance must be controlled during scale up and manufacturing once the product is approved. There is an urgent need to identify which glycoforms are the most potent and then to develop cell lines producing the optimal glycoform as homogeneously as possible.

Glycan structure considerably affects the efficacy and heterogeneity of recombinant E2 and E1E2, so it is critical to identify and control the optimal glycoforms. Mammalian-produced recombinant E2 and E1E2 both showed heterogeneous, large glycans. However, it is unlikely that these large glycans represent the actual glycosylation on the virus in vivo. While the in vivo glycoprofile of E1 and E2 have not been measured, evidence is shown here supporting that the actual in vivo glycoprofile of E1 and E2 likely contains simple glycans, and so recombinant subunit vaccines would need to be produced in a glycoengineered cell line to mimic the actual viral structure.

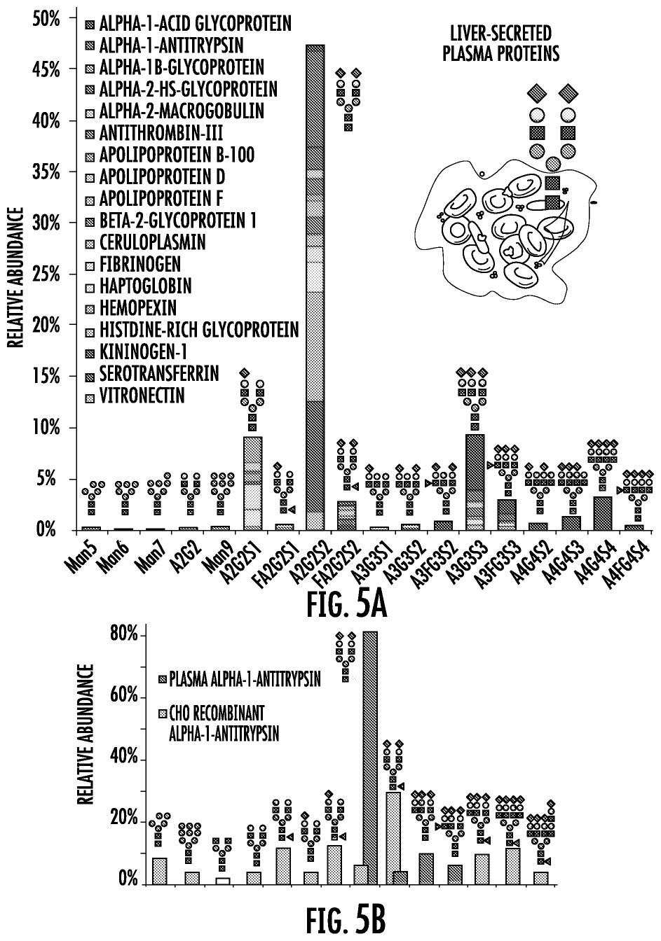

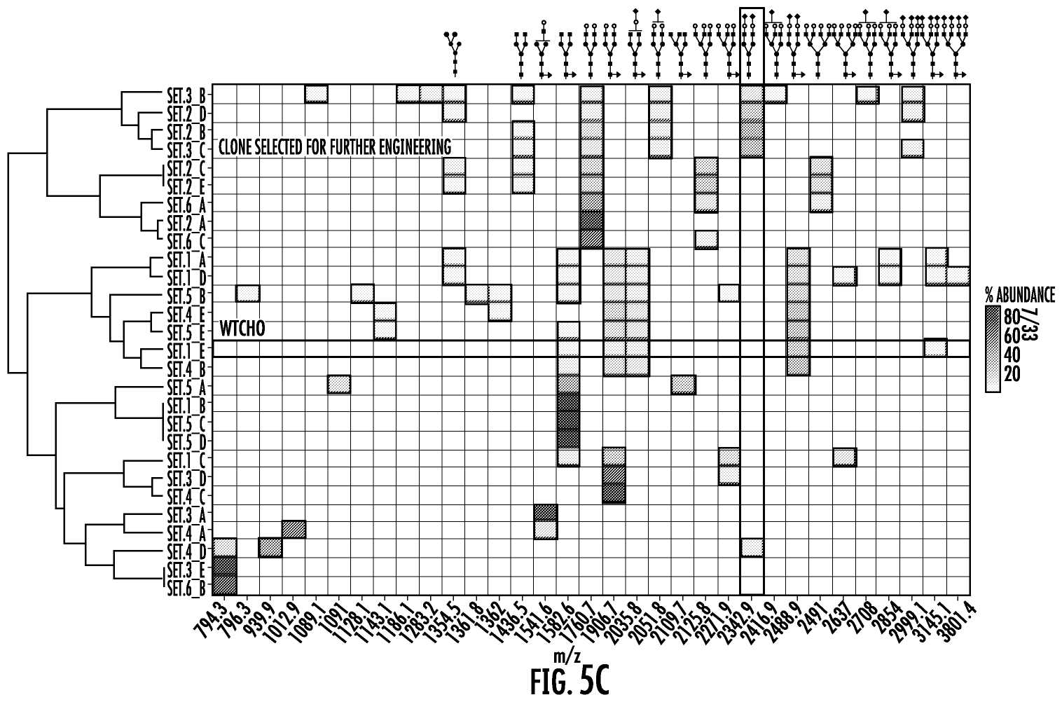

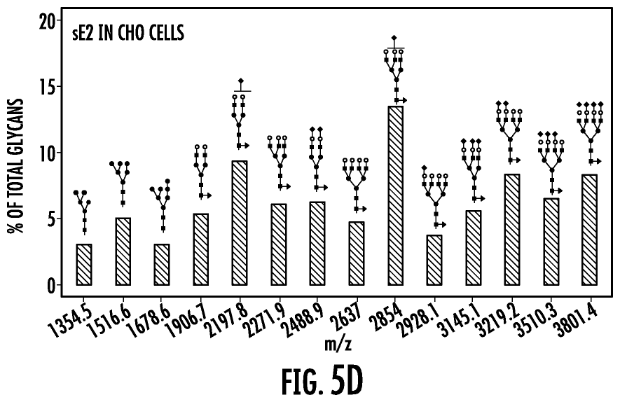

The cell type wherein HCV replicates remains debated, with evidence pointing to the liver, dendritic cells, monocytes, macrophages, B cells, bone marrow cells, oral tissues, etc. However, hepatocytes stand out as the most widely accepted candidate³⁹. Hepatocytes are also highly active protein secretion cells, and capable of secreting a vast array of glycoproteins into the blood to circulate throughout the whole body. Interestingly, the glycans on these secreted liver proteins have more simple and mostly uniform glycoprofiles (FIG. 5A), compared to what is seen from recombinant protein production, i.e., most of the secreted glycoprotein by mass, from the liver, has biantennary afucosylated glycans^{34,40}, while those same proteins, such as a-1-antitrypsin, usually express recombinantly with much larger and complex glycans (FIG. 5B). This trend holds for many liver secreted proteins and it thus is expected for HCV envelope proteins made in hepatocytes. That is, while recombinant E2 has large multi-antennary N-glycans when produced in CHO cells (FIG. 5D) and other mammalian cell lines, it is likely that more effective antibodies can be obtained from envelope proteins that are expressed in glycoengineered mammalian cells to match the glycans in size and epitopes from the host tissue. Protein produced in a library of glycoengineered CHO cells can be glycoprofiled to approximate the glycans in the host tissue, as shown for the major liver protein alpha-1-antitrypsin, when expressed in a large library of glycoengineered CHO cells (FIG. 5C).

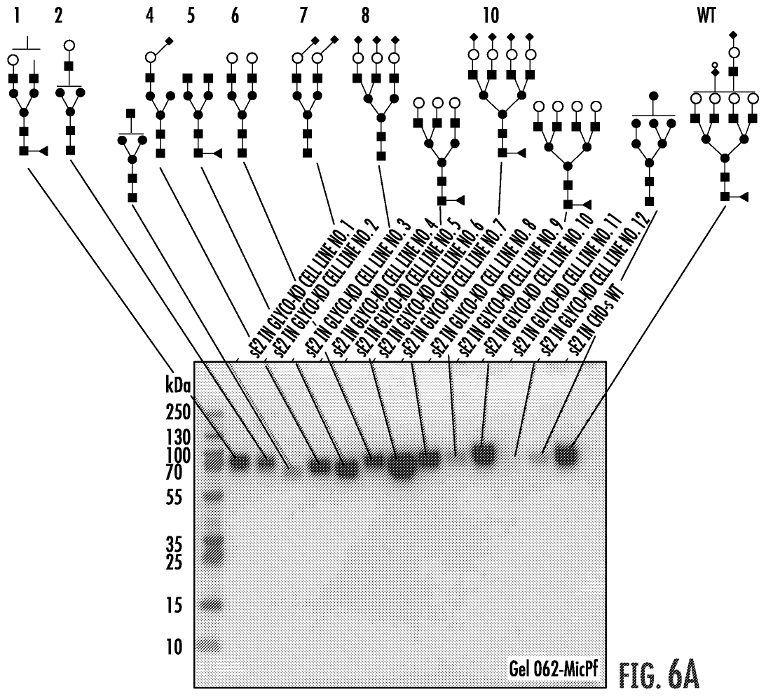

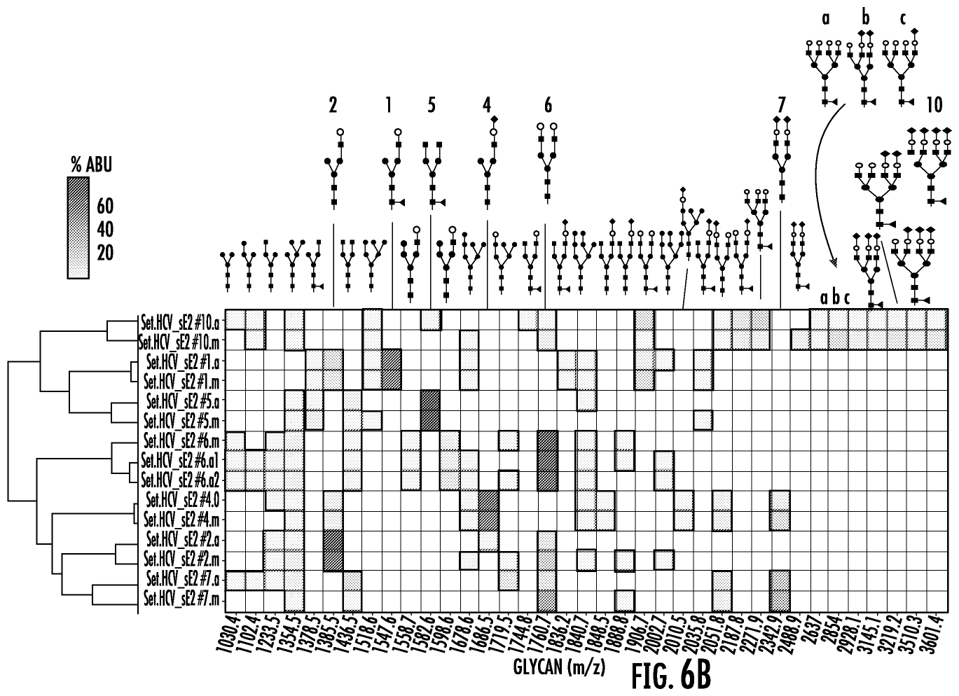

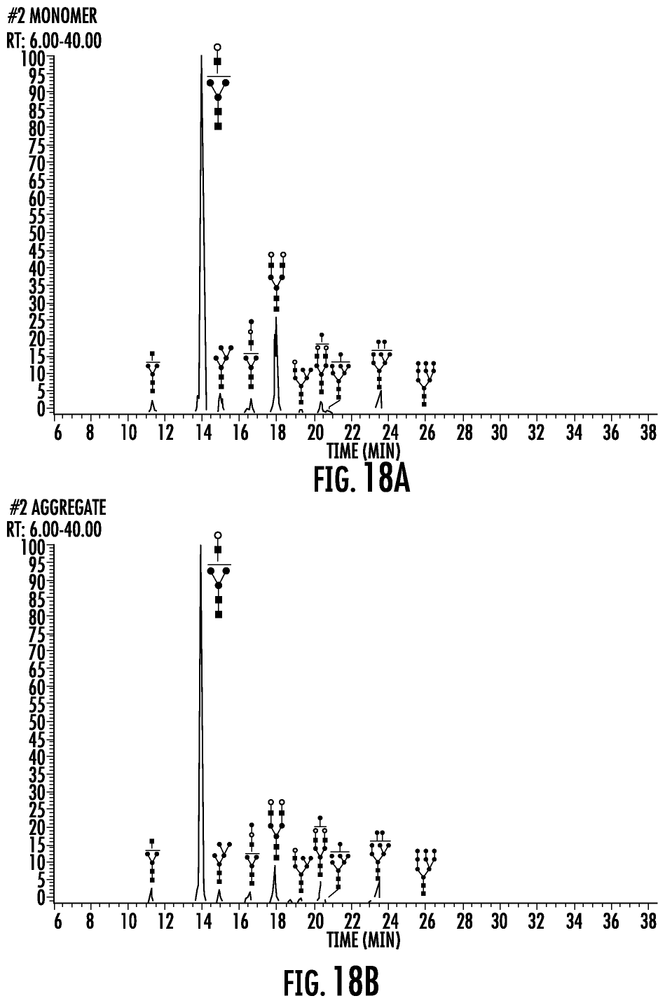

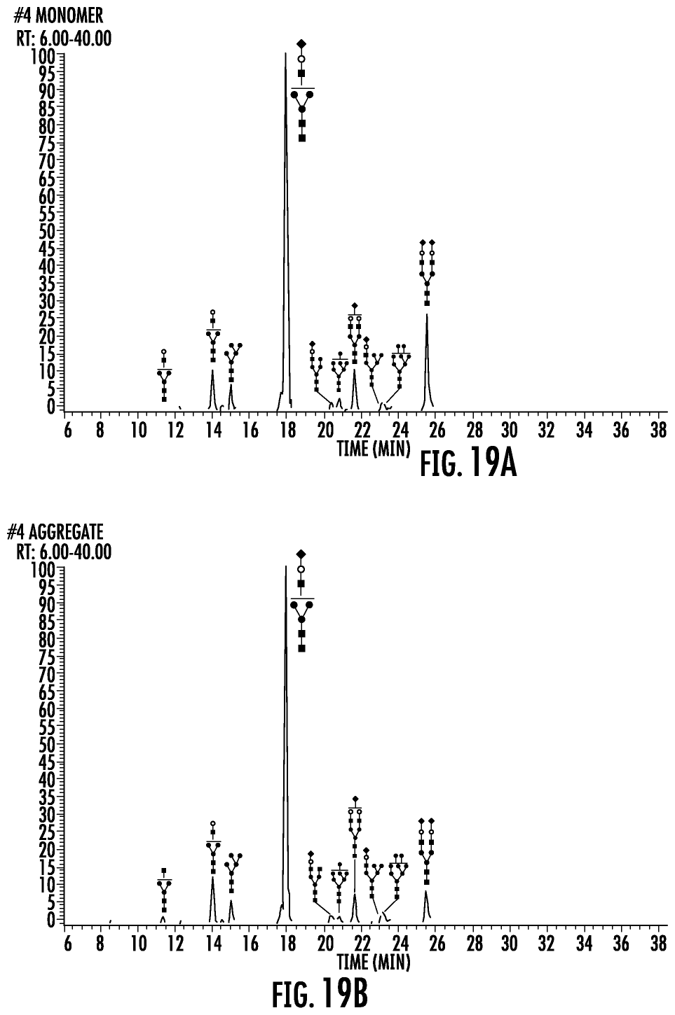

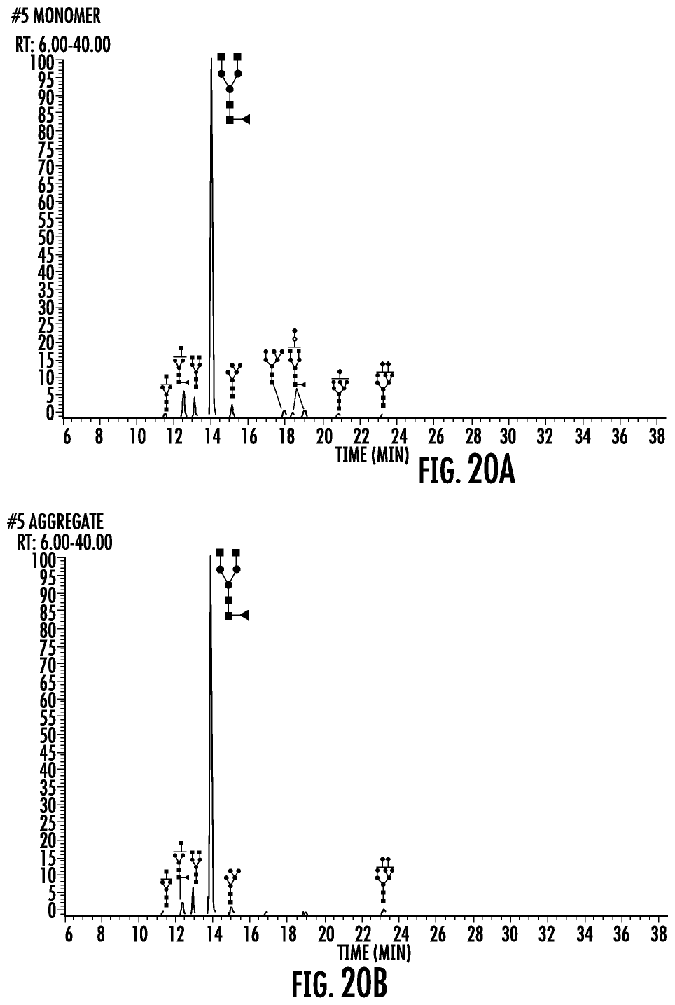

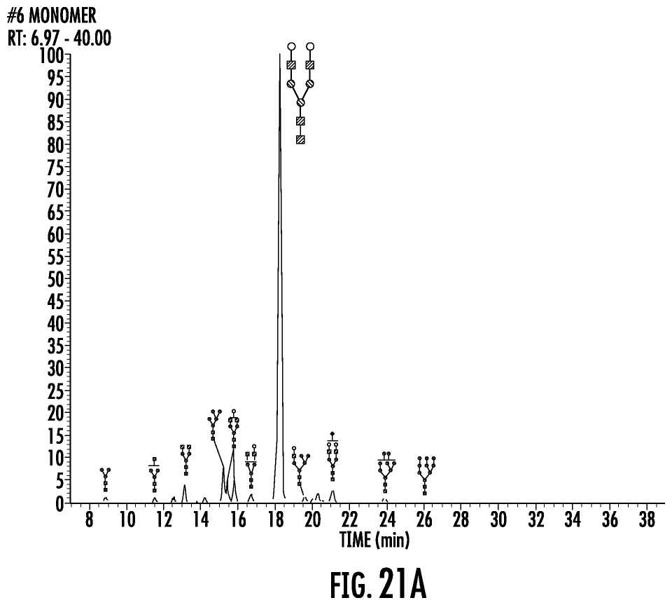

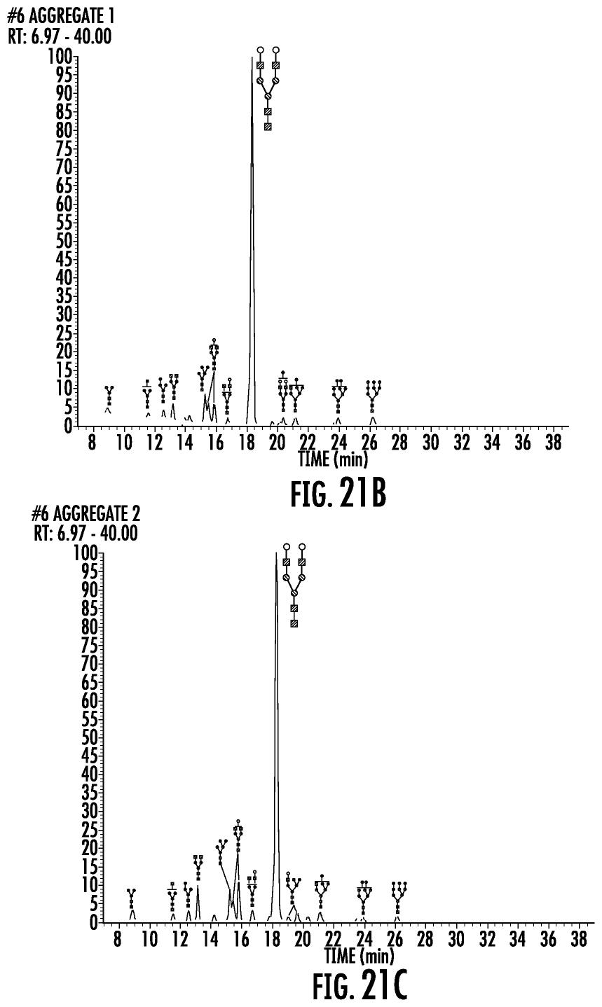

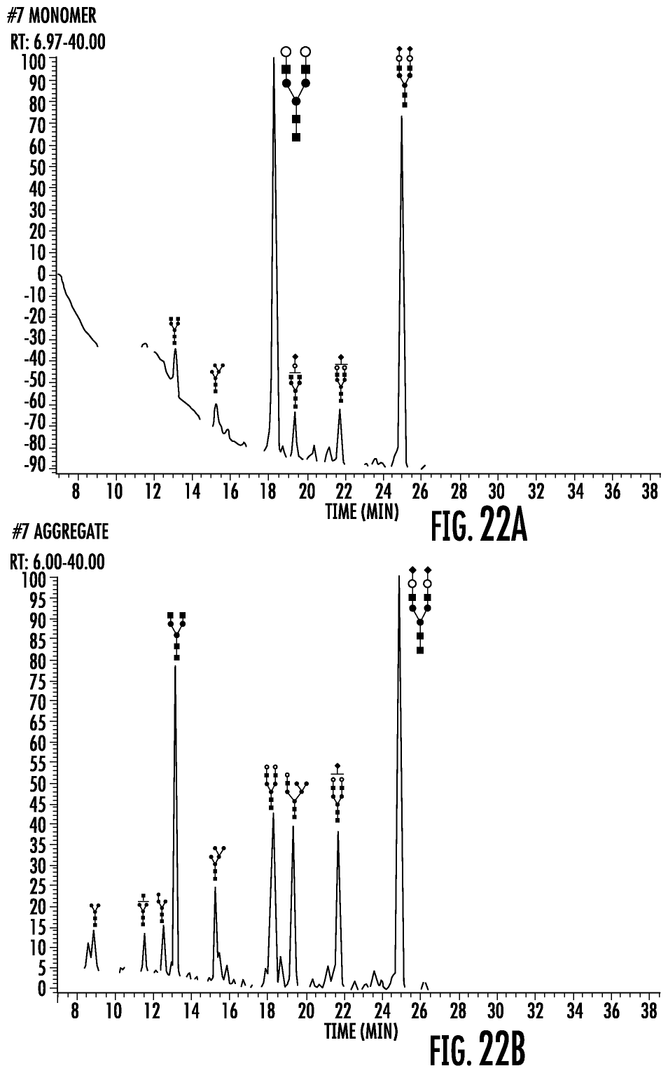

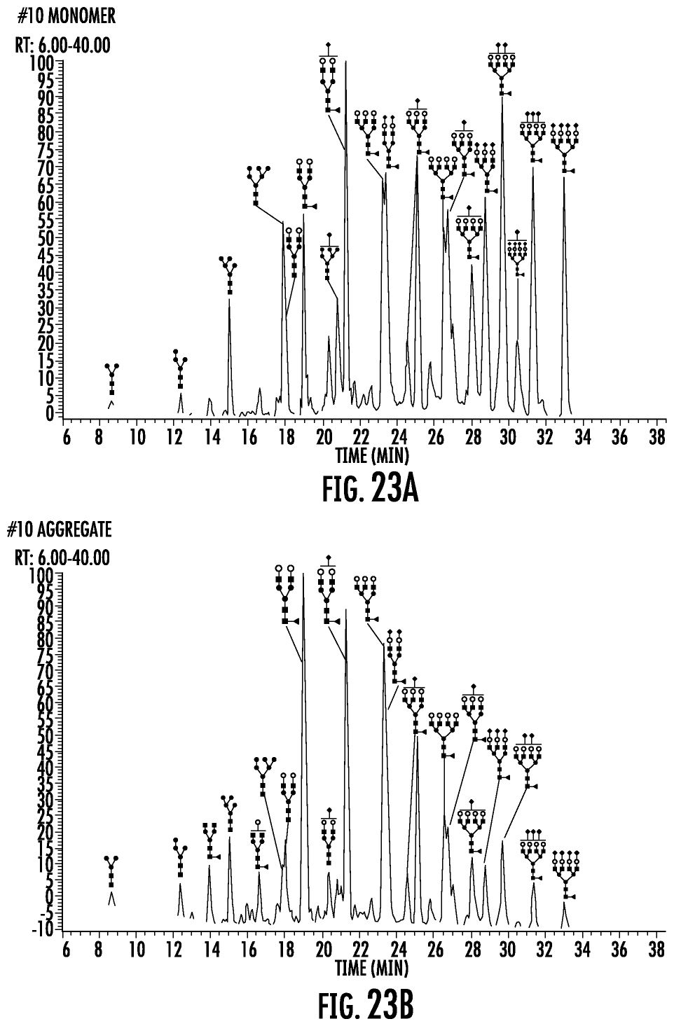

A panel of 8 glycoengineered CHO cells (termed as ‘geCHO’ cells) were tested (Table 1), wherein there were knocked out/in key genes involved in N-linked glycosylation. These cells were derived from CHO-S cells (Thermo Fisher), which can grow to industrial-level cell densities in suspension on serum-free media. Except for the #8 glycoform (wherein there was not sufficient protein produced for characterizing this glycoprofile), full glycoprofiles for the #1, #2, #4, #5, #6, #7, and #10 glycoforms are provided (FIGS. 17-23). Each mutant had a dominant glycoprofile (FIG. 6A), in which these clones express important glycoforms including fucosylated paucimannose (#1), asialylated (galactosylated) paucimannose (#2), sialylated paucimannose (#4), bi-antennary agalactosylated (#5), bi-antennary asialylated (#6), bi-antennary sialylated (#7), tri-antennary asialylated (#8), and tetra-antennary sialylated (#10) forms. Further analysis demonstrated that these cells are capable of producing diverse, but more homogeneous glycoforms of the interested sE2 recombinant proteins, each with a limited number of less abundant glycans (FIG. 6B).

TABLE 1
A panel of eight glycoengineered CHO
cells capture diverse glycoforms of sE2 protein.

| Cell line | Genotype (knock out/in glyco-genes) |
|---|---|
| geCHO sE2 #1 | st3gal3/4/6, b3gnt2, sppl3 |
| geCHO sE2 #2 | st3gal3/4/6, b3gnt2, sppl3, fut8, mgat2 |
| geCHO sE2 #4 | st3gal3/4/6, b3gnt2, sppl3, fut8, mgat2, |
| | St6Gal1(KI) |
| geCHO sE2 #5 | mgat4a/4b/5, b4galt1/2/3, b3gnt2 |
| geCHO sE2 #6 | st3gal3/4/6, b3gnt2, sppl3, fut8, mgat4a/4b/5 |
| geCHO sE2 #7 | st3gal3/4/6, b3gnt2, sppl3, fut8, mgat4a/4b/5, |
| | St6Gal1(KI) |
| geCHO sE2 #8 | b3gnt2, sppl3, fut8, mgat5 |
| geCHO sE2 #10 | b3gnt2, sppl3 |
| CHO_S WT | N.A. |

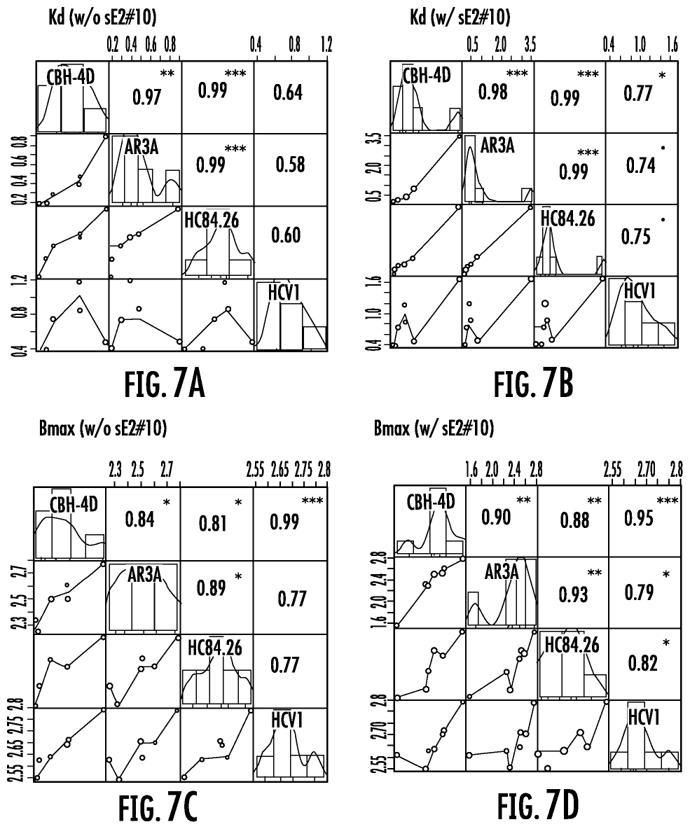

To investigate how different glycoforms might impact on the recombinant sE2 glycoprotein quality and efficacy and if those more similar to the simple glycosylation seen for liver proteins performed better, immunological testing was conducted on the bindings of different neutralizing mAbs. Table 2 shows the 10 different glycoforms of the candidate vaccine tested with 4 different neutralizing antibodies (CBH-4D, AR3A, HC84.26, and HCV1; of 40 measurements of glycoprofile-antibody pairs). CBH-4D binds domain A, AR3A binds domain B, HC84.26 binds domain D, and HCV1 binds domain E. The following summarized findings from the four assays above provide interesting insights into how glycoengineering impacts nAb binding of sE2 recombinant protein. Fit, differences in affinity of sE2 for conformation-sensitive nAbs were observed between antigens produced in WT CHO and glycoengineered cell lines, in which the cell lines #1, 2, 4, and 7 are the best, and also represent glycoforms more similar to the smaller and more simple N-linked glycans seen on typical liver-secreted proteins. Specifically, antigenic domain B (bound by AR3A) exhibits the most consistent differences, in which over 15× lower Kd relative to WT (CHO) and 8× lower relative to WT (HEK) sE2. Moreover, Bₘₐₓ also shows higher changes to WT (CHO) and WT (HEK) sE2, indicating antigenic domain B contains a more accessible epitope. Second, CBH-4D binds domain A, and the smaller glycans decrease the Kd. Interestingly, since domain A isn't glycosylated, the reduced Kd possibly resulted from the glycans elsewhere to change the conformation of domain A. Third, antigenic domain D (bound by HC84.26) showed improvement in Kds, and the glycoengineering leads to smaller glycans that enhance the Kd. Fourth, HCV1 binds domain E, and observed higher Kds on the glycoengineered sE2 expressed larger and sialylated glycans. However, the improvement in Kd over WT is only modest for the smaller glycans. Importantly the HEK glycans in domain E wouldn't be affected by most knockouts since at least one the two glycans is unprocessed and the other is already biantennary. Lastly, it was observed that three nAbs (CBH-4D, AR3A and HC84.26) show highly similar binding patterns (statistically significant correlations (spearman's correlation r>0.97, p<0.001) between nAbs; FIG. 7) to the glycoengineered sE2 proteins, suggesting that glycoengineering has a robust impact on the antigenic domains. Most importantly, these results imply that these specific glycoforms can result in a more efficacious sE2 recombinant vaccine. The best glycoforms showed 15× and 8× improved binding to neutralizing antibodies compared to the glycoforms produced in CHO-S and HEK 293 cells.

TABLE 2
Antibody binding to sE2 antigens containing either homogenous or variable glycoforms

| Kd (nM) | | | | | Bₘₐₓ | | | |
|---|---|---|---|---|---|---|---|---|
| Antigen | CBH-4D | AR3A | HC84.26 | HCV1 | CBH-4D | AR3A | HC84.26 | HCV1 |
| WT (CHO) | 0.99 | 2.86 | 1.22 | 0.69 | 2.88 | 2.12 | 2.93 | 2.87 |
| #1 | .08 | 0.18 | 0.06 | 0.4 | 2.99 | 2.78 | 2.86 | 2.84 |
| #2 | 0.10 | 0.18 | 0.12 | 0.41 | 2.8 | 2.61 | 2.69 | 2.7 |
| #4 | 0.12 | 0.28 | 0.16 | 0.75 | 2.63 | 2.33 | 2.47 | 2.55 |
| #5 | 0.26 | 0.9 | 0.28 | 0.49 | 2.71 | 2.51 | 2.73 | 2.64 |
| #6 | 0.19 | 0.38 | 0.19 | 1.18 | 2.65 | 2.26 | 2.58 | 2.63 |
| #7 | 0.19 | 0.47 | 0.2 | 0.86 | 2.8 | 2.5 | 2.68 | 2.71 |
| #8 | 0.49 | 3.43 | 0.73 | 9.57 | 2.12 | 1.57 | 2.23 | 2.63 |
| #10 | 0.68 | 3.49 | 1.00 | 1.67 | 2.35 | 1.55 | 2.41 | 2.61 |
| WT(HEK) | 0.22 | 1.47 | 0.36 | 0.34 | 2.58 | 2.01 | 2.52 | 2.81 |

ii. GEPS Identifies Key Glycans and Glycan Epitopes Impact on the nAb Binding

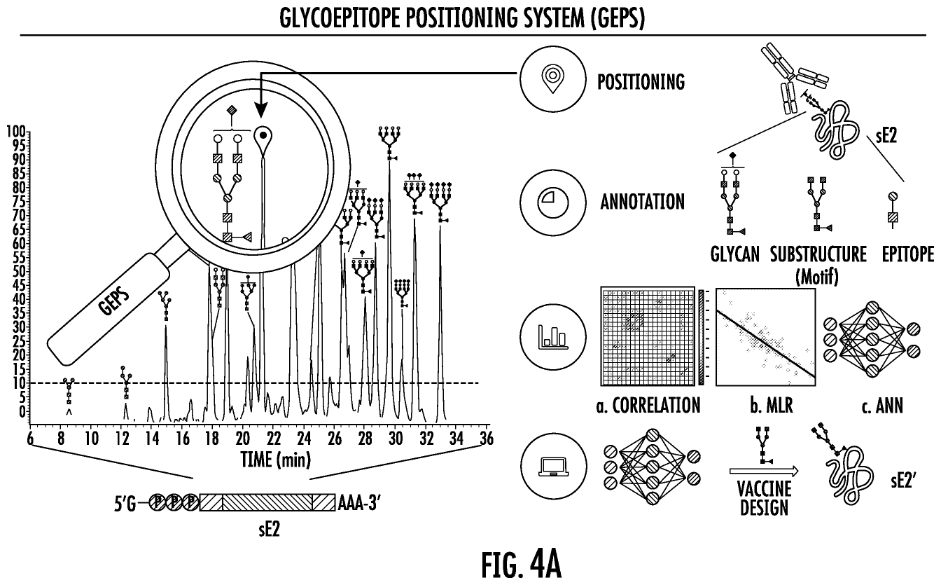

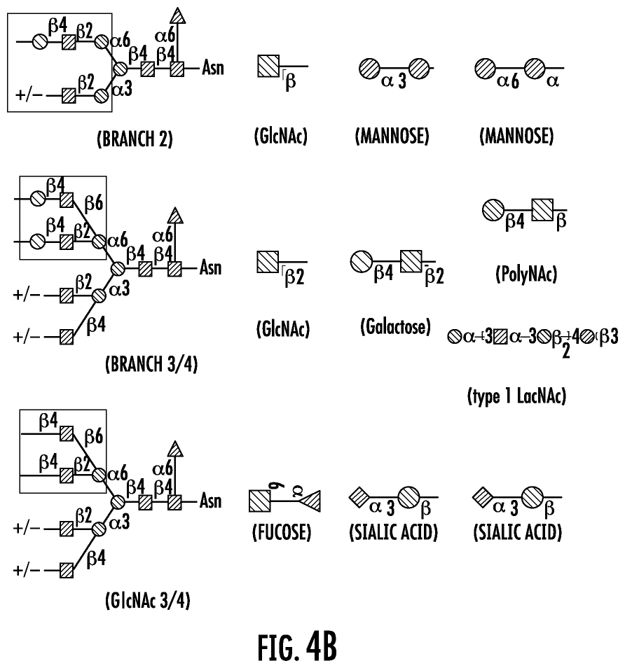

Knowing that different glycoengineered recombinant E2 vaccine candidates led to different nAb responses, the question of what are the optimal glycan structures for nAb binding remained? The reason to identify the optimal glycoform for nAb binding is to use further clone selection, media optimization, and/or cell engineering. To achieve this, the glycoinformatics pipeline—GlycoEpitope Positioning System (GEPS)—was developed to link glycans or glycan epitopes to product quality and function (FIG. 4). Specifically, the GEPS system takes the glycanprofiles and decomposes them to glycan epitopes for each glycan. Thirteen structural features of N-glycans (FIG. 4B and Table 3) are examined, which cover major glycan epitopes on mammalian N-glycan. The GEPS system then determines directionality and strength of the relationship between individual glycan/epitope and nAb binding (Kd/Bmax) or any other preclinical or clinical feature.

TABLE 3
Selected glycan epitopes and their binding lectins for N-glycan characterization

| | | Sugar binding | |
|---|---|---|---|
| Lectin | Name | specificity*^{a} | Recognition Logic*^{b} |
| PHA-E | Phaseolus vulgaris | Bisecting GlcNAc and | At least one exposed |
| | Erythroagglutinin | biantennary N-glycans | ‘GlcNAc’ on the branch 2. |
| PHA-L | Phaseolus vulgaris | Tri-/Tetra-antennary | Branch = 3 or 4; bisecting |
| | Leucoagglutinin | complex-type N-glycans | GlcNAc (if any) |
| AOL | Aspergillus oryzae | Fucose | ‘(Fa6)GN’ |
| GNA | Galanthus nivalis | α-Man | ‘(Ma3Ma’; ‘)Ma3Ma’ |
| | Agglutinin | | |
| NPA | Narcissus | Non-substituted α1- | ‘(Ma6Ma’; ‘)Ma6Ma’ |
| | pseudonarcissus | 6Man | |
| | Agglutinin | | |
| MAH | Maackia amurensis II | Siaα2-3Gal | ‘(NNa3Ab’; ‘)NNa3Ab’ |
| SNA | Sambucus nigra | Siaα 2-6 Galβ1- | ‘(NNa6Ab’; ‘)NMa6Ab’ |
| | Agglutinin | 4Glc(NAc) | |
| STL | Solanum Tuberosum | Poly-LacNAc and | ‘(Ab4GNb’; ‘)Ab4GNb’ |
| | Lectin | (GlcNAc)n | |
| Galectin-7 | Galectin-7 | Galβ1-3Glc(NAc) | ‘(Ab4GNb3’;‘)Ab4GNb3’ |
| | | (type 1 LacNAc) | |
| GSL-II | Griffonia simplicifolia | GlcNAc and | At least one exposed |
| | II | agalactosylated | ‘GlcNAc’ on the branch 3 or |
| | | N-glycans | 4. |
| Nictaba | Nicotiana tabacum | GlcNAc | ‘(GNb’; ‘)GNb’ |
| | agglutinin | | |
| RCA120 | Ricinus communis I | Galβ1-4Glc(NAc) | ‘(Ab4GNb2’;‘)Ab4GNb2’ |
| WGA | Triticum unlgari | Multivalent Sia and | ‘(GNb2’;‘)GNb2’ |
| | | (GlcNAc)ₙ | |

*^{a}The sugar abbreviations of ‘Fuc’, ‘Gal’, ‘GalNAc’, ‘Glc’, ‘GlcNAc’, ‘Man’, and ‘Sia’ represent L-Fucose, D-Galactose, N-Acetylgalactosamine, D-Glucose, N-Acetylglucosamine, Mannose, and Sialic Acid respectively.
*^{b}Recognition logic is the rule that was used to detect if a given glycan in a MS glycoprofile contains the specific glycan structure that can be bound by an indicated lectin. The abbreviations of ‘A’, ‘F’, ‘GN’, ‘M’, and ‘NN’ represent galactose, fucose, GlcNAc, mannose, and NAcNAc respectively, whereas ‘aX’ or ‘bX’ (where ‘X’ is a number) represents an alpha or beta glycosidic bond connecting the two adjacent sugars (e.g. a3 represents alpha 1,3 glycosidic bond).

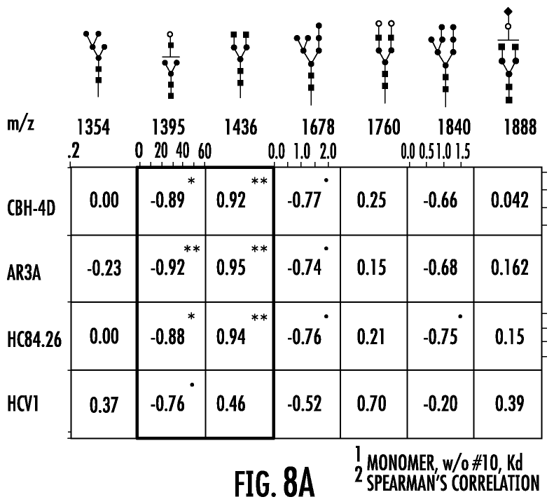

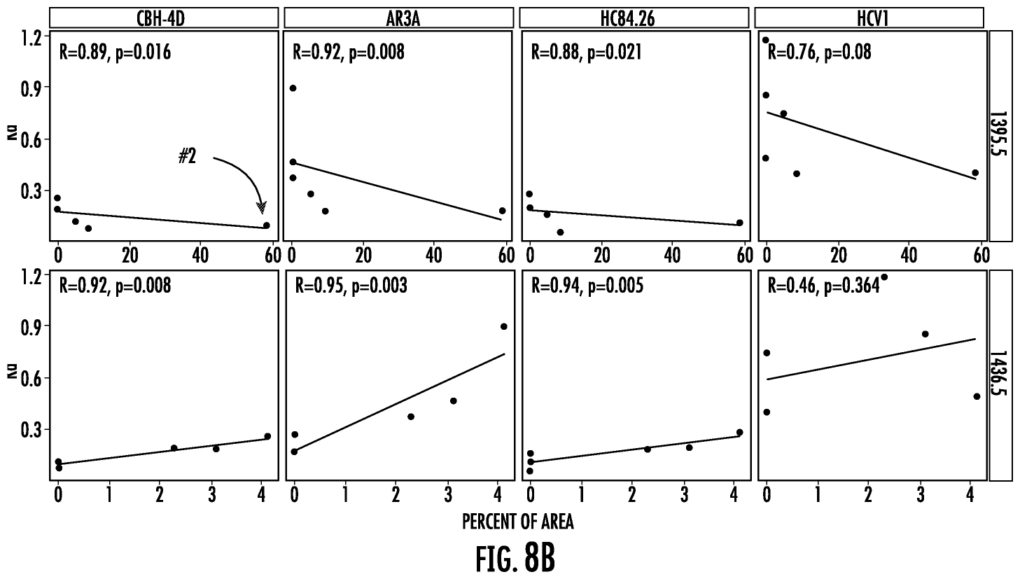

The GEPS system identified two glycans that strongly correlate with nAb binding Kd (FIG. 8). The first glycan (m/z=1395) is an asialylated (galactosylated) paucimannose glycan, which shows a strong negative correlation (Spearman's correlation: r<−0.88, p<0.05) with Kd for nAb binding of AR3A, CBH-4D, and HC84.26. Apparently, the more galactosylated paucimannose glycan expressed, the lower the Kd (higher nAb binding affinity) will be. Indeed, this glycan is the main glycan structure expressed in the geCHO #2, in which it showed the lowest Kd for nAb binding of AR3A and the 2nd lowest Kd for CBH-4D and HC84.26. However, the glycan is also observed in lower amounts in products from cell lines #1 and #4. The second identified glycan (m/z=1436) is a bi-antennary agalactosylated glycan, which shows a strong positive correlation (Spearman's correlation: r>0.92, p<0.01) with K_{d} values for nAb binding of AR3A, CBH-4D, and HC84.26. It indicates that the more bi-antennary agalactosylated glycan expressed, the higher the K_{d} (lower nAb binding affinity) will be. This glycan is the main glycan structure expressed in the geCHO #5, in which it showed the highest K_{d} for the binding of nAbs (AR3A, CBH-4D, and HC84.26), but also found in geCHO #7, potentially decreasing the potency of its product.

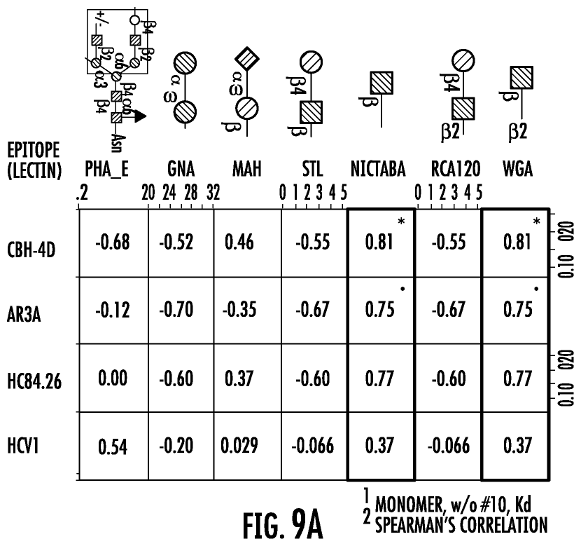

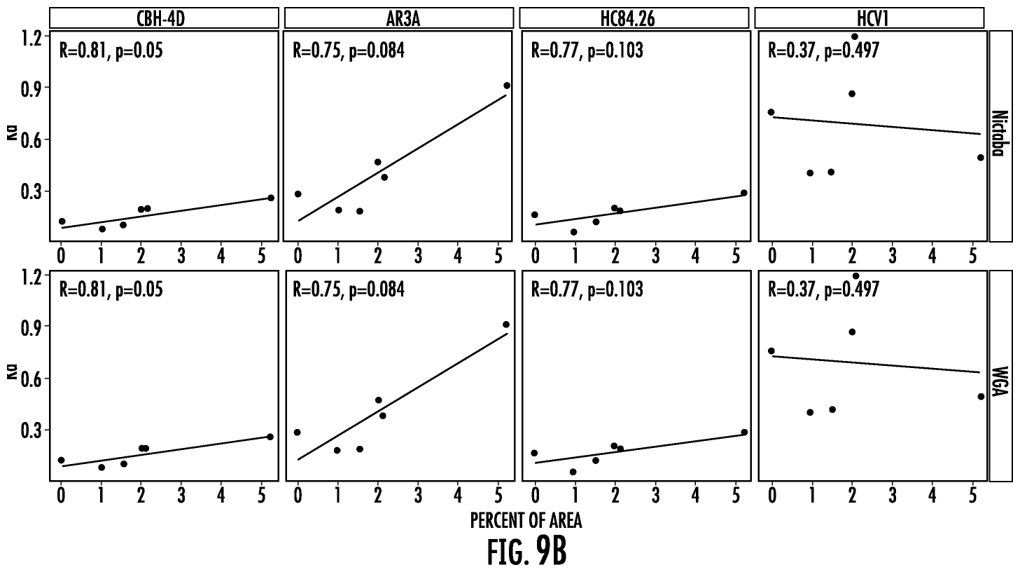

The GEPS system also identified two specific glycan epitopes that strongly correlate with nAb binding K_{d} (FIG. 9). One is the exposed GlcNAc (recognized by Nicotiana tabacum agglutinin (Nictaba) lectin), and the other one is the internal GlcNAc on the β2 linkage (recognized by the wheat-germ agglutinin (WGA) lectin). Both of these two glycan epitopes showed a significant positive correlation (Spearman's correlation: r>=0.75, p<0.05) with K_{d}s for nAb binding of AR3A and CBH-4D. It indicates that the more exposure or internal GlcNAc antigen, the higher the K_{d} (lower nAb binding affinity) will be. Interestingly, these two glycan features are actually the antigens of the above identified bi-antennary agalactosylated glycan. Another notable finding is the moderate negative correlation (Spearman's correlation: r<−0.5) with K_{d} for the three glycan epitopes: exposed mannose (recognized by GNA lectin), galactose on the β2 linkage (RCA120 lectin), and poly-LacNAc (STL lectin). While they are individually not statistically significant (p>0.05), it implies that these three glycan epitopes are associated with lower K_{d} values (higher nAb binding affinity). One possible explanation is that these three antigens might work combinatorial for more efficacious nAb binding. Interestingly, the galactose on the β2 linkage and exposed mannose are indeed the glycan features of the above identified asialylated (galactosylated) paucimannose glycan (m/z-1395).

In summary, these results provide a global overview of all glycoengineered CHO cells, allowing for determination of optimal glycans and epitopes for downstream clone selection. Specifically, the vaccine design prefers the asialylated (galactosylated) paucimannose glycan (m/z=1395) and should avoid the bi-antennary agalactosylated glycan (m/z=1436). Moreover, the favored glycan antigens for the vaccine design are the β2-linked galactose (RCA120 lectin) and exposed mannose (GNA lectin), but the disfavored glycan antigens are the exposed or β2-linked GlcNAc (WGA lectin).

iii. GEPS Enables to Predict nAb Binding Strength from Glycan Epitope by Using Multinomial Linear Regression Model

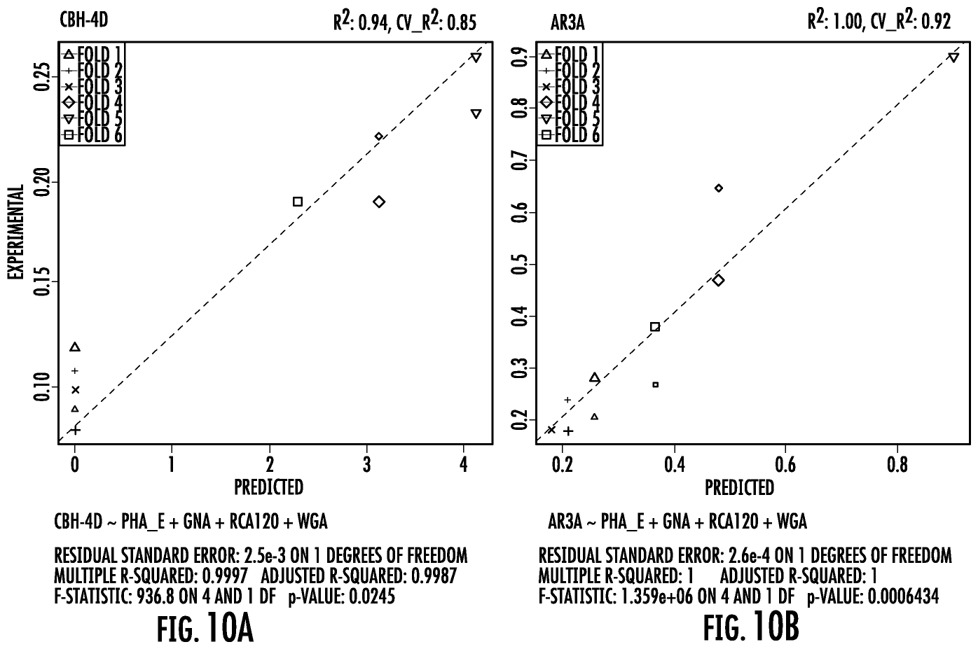

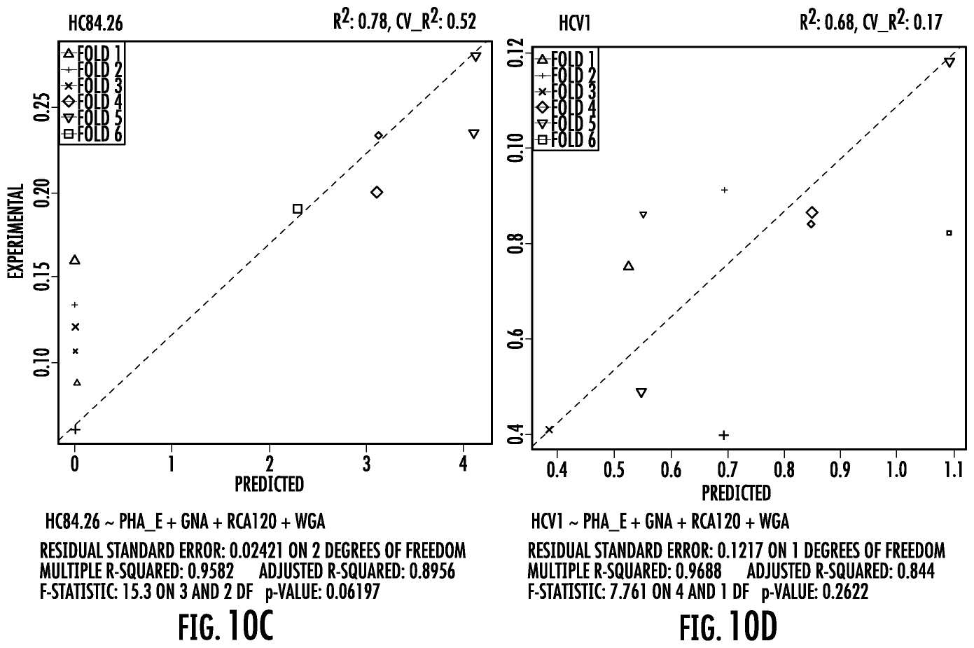

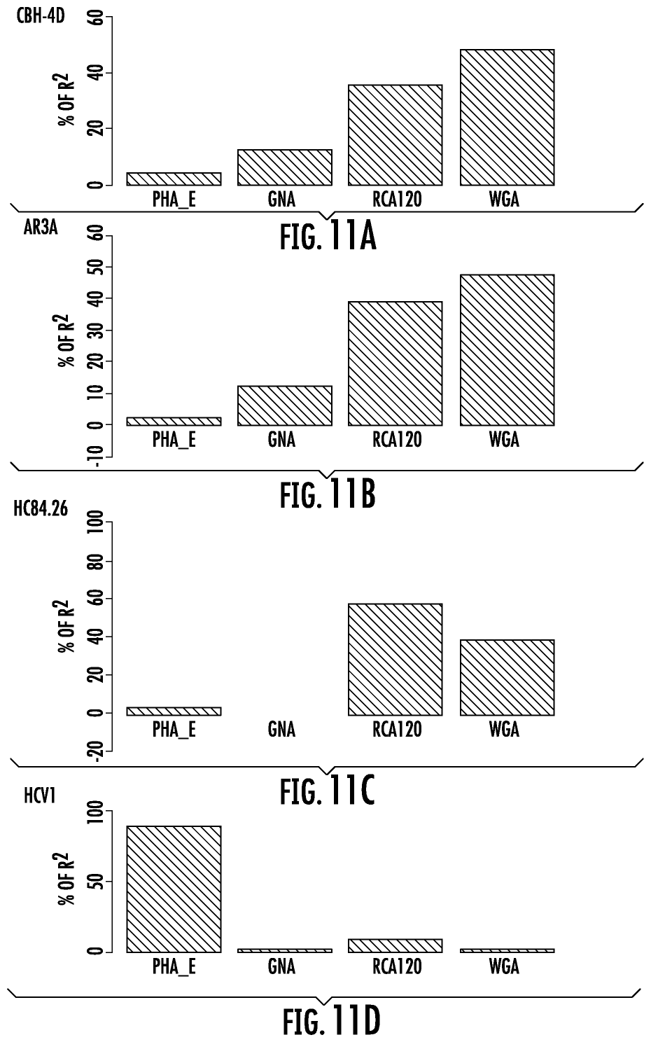

To gain insights on whether the glycan antigens can work cooperatively for achieving optimal nAb binding efficacy, the GEPS system employed machine learning algorithms for identifying the best combination of glycan antigens for modelling the relationship between glycan epitope abundance and nAb binding. Specifically, multinomial linear regression^{41,42} was employed to understand how nAb binding (dependent variable) changes when the abundance of glycan antigens (independent variables) change. The leave-one-out cross validation was applied, wherein the learning process was applied once for each geCHO sample, using all other geCHO samples as a training set and using the selected geCHO sample as a single-item test set.^{43,44} The results (FIG. 10 and Tables 4-7) show strong prediction of CBH-4D (training R²=0.94 and cross-validation (CV) R²=0.85; p-value=0.0245) and AR3A (training R²=1.00 and CV_R²=0.92; p-value=6.43e-04) nAb binding K_{d}. However, there was weaker prediction performance on the HC84.26 (training R²=0.78 and CV_R²=0.52; p-value=6.19e-02) and HCV1 (training R²=0.68 and CV_R²=0.17; p-value=0.2622) nAbs. These results indicate that multiple glycan epitopes might synergistically work and account for the interaction with the CBH-4D and AR3A nAbs. To further understand the importance of the modeled glycan epitopes, the variance was quantified that could be explained by these glycan antigens using the Correlation-Adjusted coRelation (CAR) scores.³⁶ The results (FIG. 11) show that the exposed mannose (GNA lectin) and β2-linked galactose (RCA120 lectin) account for around 50% of the Kd binding variance of CBH-4D (12.3% and 35.4% respectively) and AR3A (12.2% and 38.6% respectively) nAbs, and the β2-linked GlcNAc (WGA lectin) account for another ˜50% of the Kd binding variance of CBH-4D (48.3%) and AR3A (47.3%) nAbs. However, β2-linked GlcNAc (WGA lectin) positively correlates with Kd, but the exposed mannose (GNA lectin) and β2-linked galactose (RCA 120 lectin) positively correlate with Kd (FIG. 9 and Table 4). Another interesting finding is that β2-linked galactose (RCA 120 lectin) and GlcNAc (WGA lectin) accounted for >96% of the Kd binding variance for the HC84.26. Surprisingly, for the HCV1, the biantennary N-glycan antigen (PHA_E lectin) accounted for ˜90% of the Kd binding variance. All these results thus provide a comprehensive picture on how multiple glycan antigens can contribute to the variation of the nAb binding, in which the derived optimal glycoforms can be used for further preclinical studies to further validate their antigenic role of the vaccine.

TABLE 4
Model #1: CBH-4D~PHA_E + GNA + RCA120 + WGA

| | | Std. | | | Signif. |
|---|---|---|---|---|---|
| | Estimate | Error | t value | Pr(>\|t\|) | codes |
| (Intercept) | 0.148 | 0.010 | 14.441 | 0.044 | * |
| PHA_E | 0.041 | 0.002 | 26.429 | 0.024 | * |
| GNA | −0.003 | 0.003 | −0.988 | 0.503 | |
| RCA120 | −0.033 | 0.001 | −27.573 | 0.023 | * |
| WGA | 0.022 | 0.001 | 27.133 | 0.024 | * |

Signif. codes:
’***’: p < 0.001,
’**’: p < 0.01,
‘*’: p < 0.05

Residual standard error: 0.002495 on 1 degrees of freedom
Multiple R-squared: 0.9997
F-statistic: 936.8 on 4 and 1 DF

TABLE 5
Model #2: AR3A~PHA_E + GNA + RCA120 + WGA

| | | Std. | | | Signif. |
|---|---|---|---|---|---|
| | Estimate | Error | t value | Pr(>\|t\|) | codes |
| (Intercept) | 6.54E−01 | 1.07E−03 | 612 | 0.001 | ** |
| PHA_E | 5.75E−02 | 1.62E−04 | 354.6 | 0.002 | ** |
| GNA | −7.89E−02 | 3.48E−04 | −226.4 | 0.003 | ** |
| RCA120 | −9.29E−02 | 1.24E−04 | −752.4 | 0.001 | *** |
| WGA | 8.44E−02 | 8.79E−05 | 960.1 | 0.001 | *** |

Signif. codes:
’***’: p < 0.001,
’**’: p < 0.01,
‘*’: p < 0.05

Residual standard error: 0.0002597 on 1 degrees of freedom
Multiple R-squared: 1
F-statistic: 1.359e+06 on 4 and 1 DF

TABLE 6
Model #3: HC84.26~PHA_E + RCA120 + WGA

| | | Std. | | | Signif. |
|---|---|---|---|---|---|
| | Estimate | Error | t value | Pr(>\|t\|) | codes |
| (Intercept) | 0.194 | 0.037 | 5.264 | 0.034 | * |
| PHA_E | 0.047 | 0.013 | 3.495 | 0.073 | |
| RCA120 | −0.044 | 0.011 | −4.152 | 0.053 | |
| WGA | 0.017 | 0.008 | 2.129 | 0.167 | |

Signif. codes:
’***’: p < 0.001,
’**’: p < 0.01,
‘*’: p < 0.05

Residual standard error: 0.02421 on 2 degrees of freedom
Multiple R-squared: 0.9582
F-statistic: 15.3 on 3 and 2 DF

TABLE 7
Model #4: HCV1~PHA_E + GNA + RCA120 + WGA

| | | Std. | | | Signif. |
|---|---|---|---|---|---|
| | Estimate | Error | t value | Pr(>\|t\|) | codes |
| (Intercept) | 0.217 | 0.501 | 0.432 | 0.740 | |
| PHA_E | 0.397 | 0.076 | 5.216 | 0.121 | |
| GNA | 0.164 | 0.163 | 1.007 | 0.498 | |
| RCA120 | −0.219 | 0.058 | −3.790 | 0.164 | |
| WGA | −0.027 | 0.041 | −0.655 | 0.631 | |

Signif. codes:
’***’: p < 0.001,
’**’: p < 0.01,
‘*’: p < 0.05

Residual standard error: 0.1217 on 1 degrees of freedom
Multiple R-squared: 0.9688
F-statistic: 7.761 on 4 and 1 DF

iv. GEPS Predicts nAb Binding Strength from Glycan Epitope by Using a Neural Network Model

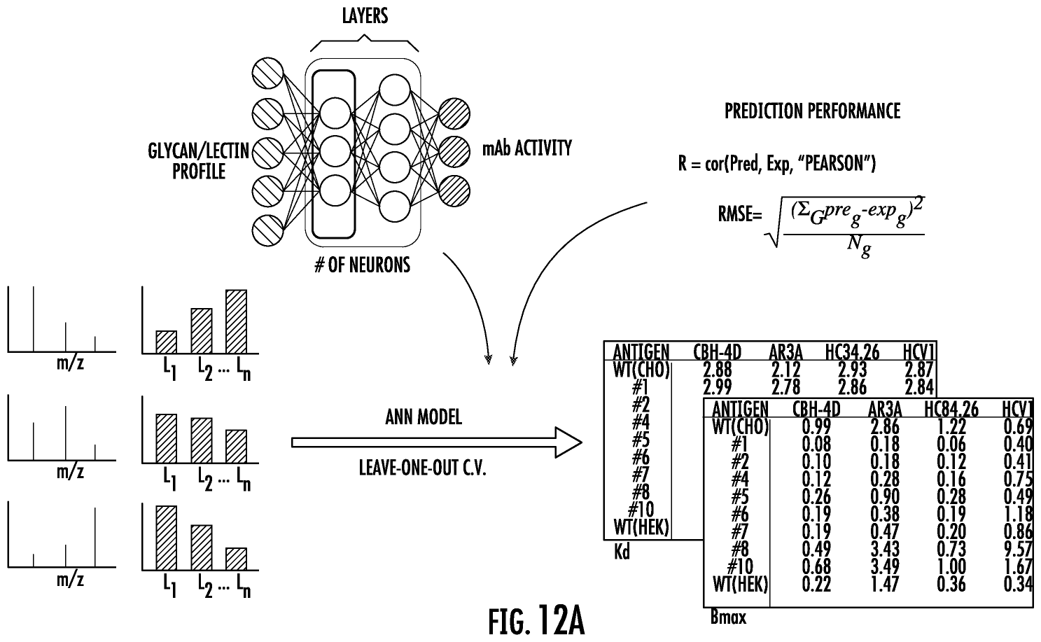

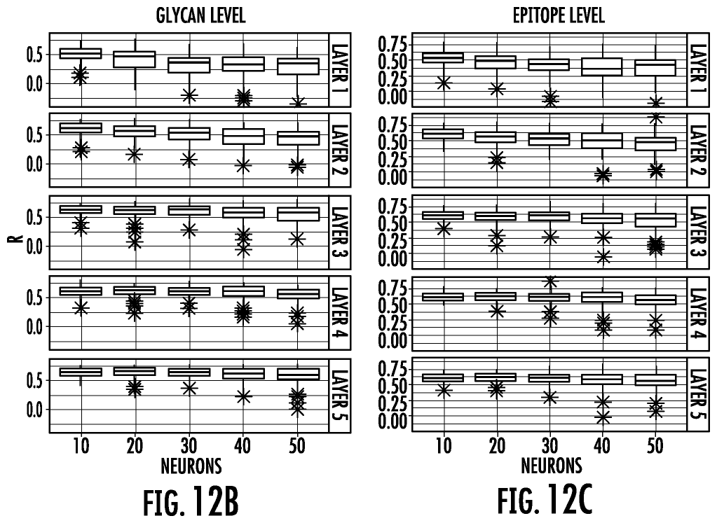

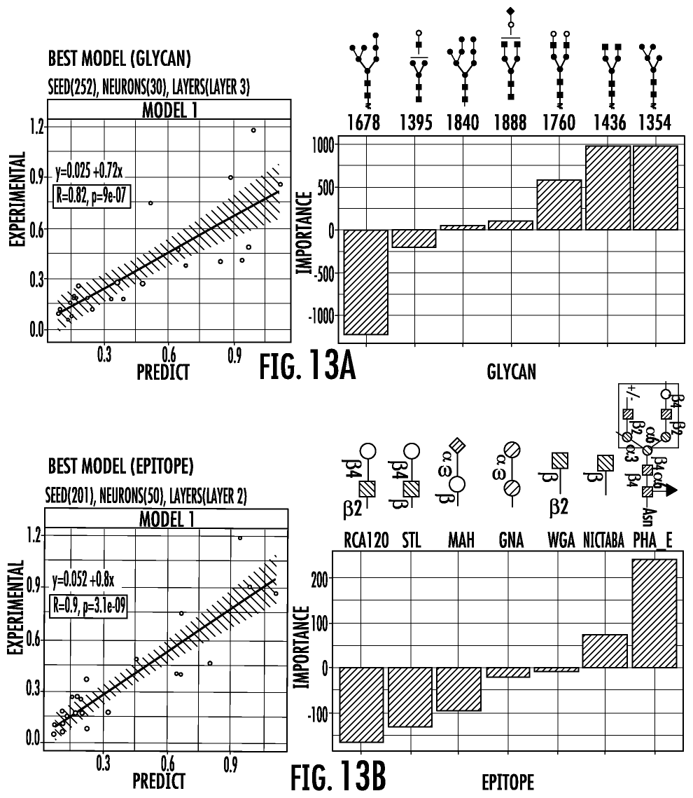

nAb binding can be predicted from glycan/epitope profiles after learning a neural network model from the glycoengineered subunit vaccines. The GEPS system was further developed to train a neural network model that can take any glycan or epitope profile and make predictions on binding affinity for each nAb of interest (FIG. 12A). A typical neural network consists of one or more hidden layers, and the prediction performance is influenced by the neural network topology. Therefore, the first step is to determine the optimal neural network topology. Different combinations of hidden layer size and neuron size in each layer were tried (FIGS. 12B-C). Based on the leave-one-out cross-validation, FIG. 13 shows the best neural network models with excellent prediction performance for the four assayed nAbs. For the best NN_{glycan} model, the neural network has three hidden layers and each layer has 30 neurons with excellent prediction performance (R=0.82, p<9e-07) (FIG. 13A). For the best NNₑₚᵢₜₒₚₑ model, the neural network uses different network structures (two hidden layers and each layer has 50 neurons) but also presents great prediction performance (R=0.90, p<3.1e-09) (FIG. 13B). Furthermore, the prediction performance of the best NNₑₚᵢₜₒₚₑ model is 8% better than the best NN_{glycan} model, which indicates that epitope features can capture better nAb binding information than glycan features. To further understand the importance of input glycans or epitopes in neural networks, the relative importance of each lectin is quantified as the sum of the product of raw input-hidden and hidden-output connection weights between each input and output neuron and sums the product across all hidden neurons.^{45,46} Consistent with the previous analysis (FIG. 9), the NNₑₚᵢₜₒₚₑ model indicates epitopes of exposed GlcNAc (Nictaba and PHA_E) are positively associated with nAb binding K_{d}, and the exposed galactose (RCA120 and STL), could enhance nAb binding (decreased K_{d}). Also, the results are in line with FIG. 8 and indicate that the bi-antennary agalactosylated glycan (m/z=1436) is positively associated with nAb binding K_{d}, and the galactosylated paucimannose glycan (m/z=1395) could enhance nAb binding (decreased K_{d}). The insights here can be further used for designating recombinant vaccine antigens with the optimal nAb binding efficacy.

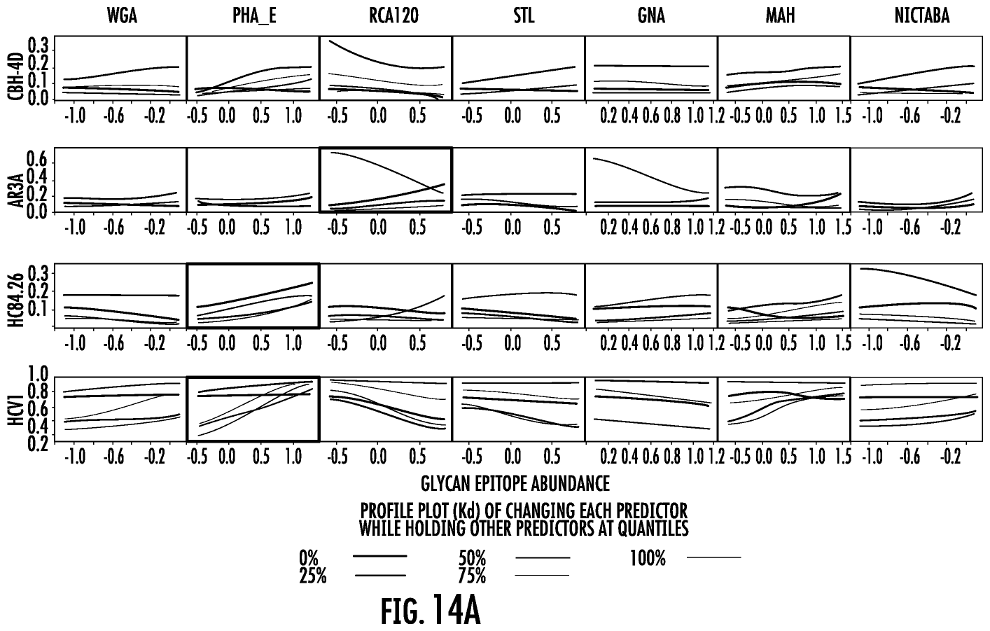

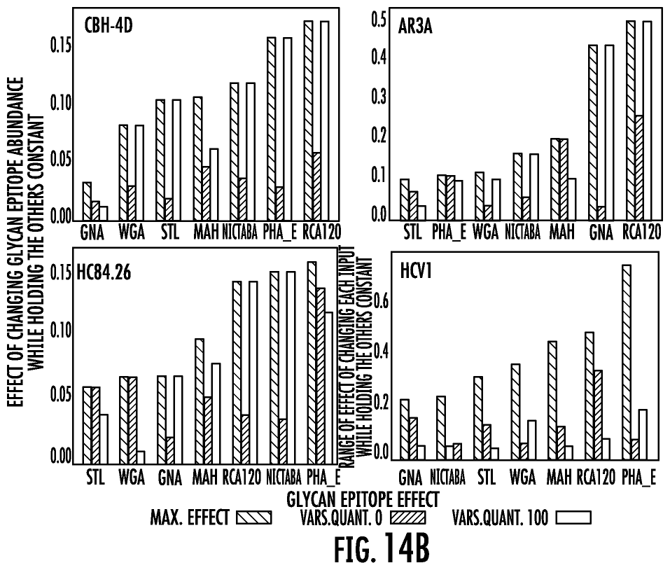

The relationships between a response variable (nAB binding K_{d}) with the explanatory variables (epitope abundance) were used in the best NNₑₚᵢₜₒₚₑ model. Specifically, how does a response nAB binding K_{d} change in relation to increasing or decreasing values of a given glycan epitope was asked? To address this question, the sensitivity analysis⁴⁵⁻⁴⁷ of the NNₑₚᵢₜₒₚₑ model was performed. Wherein, the K_{d} profile was investigated by changing each epitope while holding others constant at their specified quantiles (the minimum (dark blue), 25^{th} (blue), 50^{th} (light blue), 75^{th} (light grey), and maximum (grey) quantile values) (FIG. 14A). The results show that changes in the exposed galactose (RCA120) had its maximum effect on the AR3A and CBH-4D nAb bindings, but maximum effects of change the bi-antennary exposed GlcNAc (PHA_E) would lead to most varied effect on the HC84.26 and HCV1 nAb bindings (FIGS. 14A-B). The sensitivity analysis therefore provides valuable information on the relationship between a nAb and an epitope of interest, but the effect can be impacted by abundance of other epitopes (even linear or non-linear interactions may be present). The identified epitopes with max effects on the nAbs are promising candidates to be considered for engineering the CHO cells or optimizing glycosylation through process optimization, in order to improve the potency of a recombinant subunit vaccine.

v. GEPS Enables In-Silico Clone Screening for the Optimal Glycoform of Recombinant Vaccine Using Neural Network Model

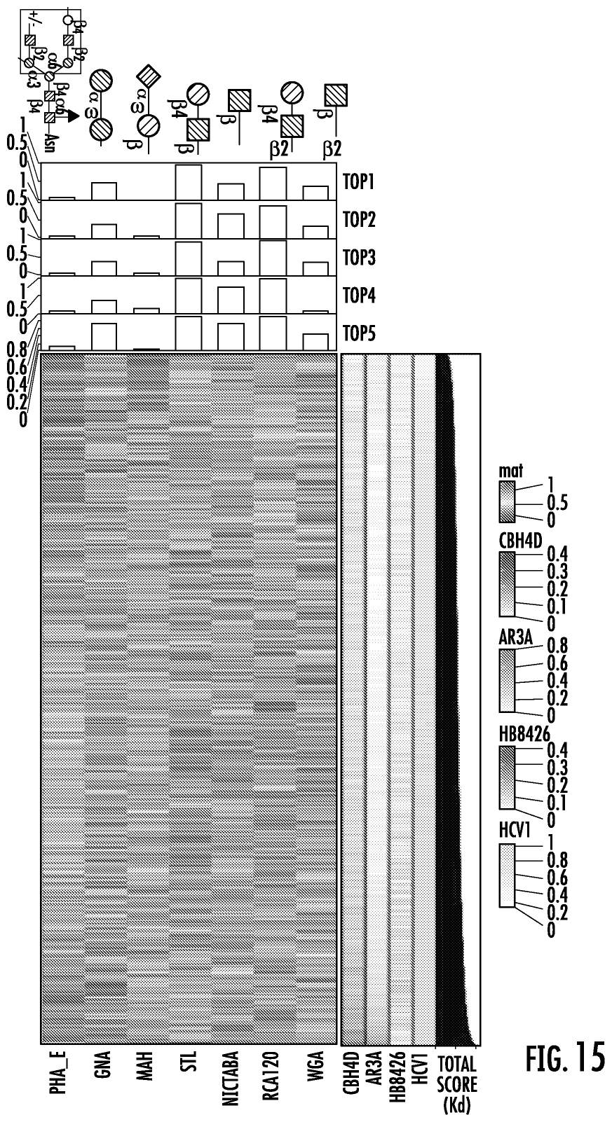

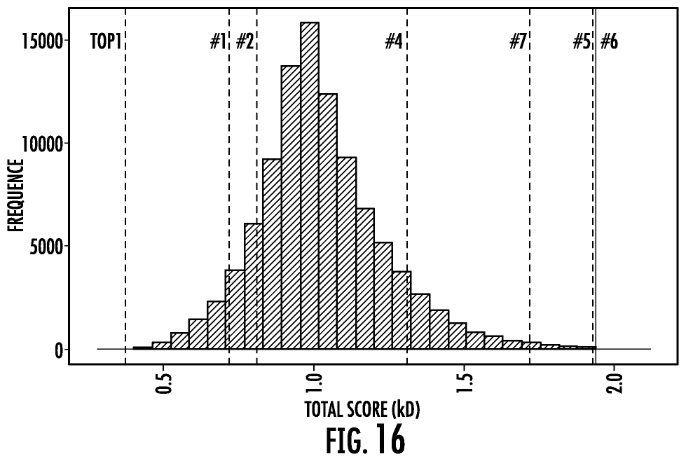

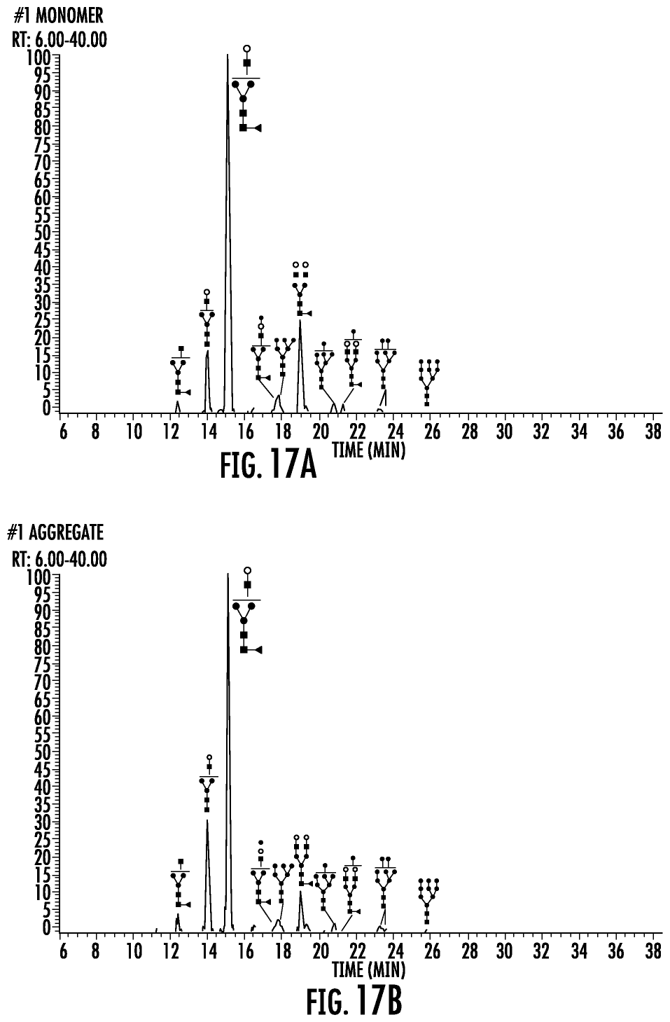

Lastly, it was explored whether the NN model can be used for in-silico clone screening the optimal glycoform that minimized the nAb K_{d}. Compared to the sensitivity analysis that just changes one epitope at a time, this analysis predicts the optimal glycoforms for additional glycoengineering to the host cells. To achieve this, 10⁵ different combinations of epitope profiles (termed as NN-geCHO samples) were sampled using the Latin Hypercube Sampling (LHS) algorithm³⁷. The best NNₑₚᵢₜₒₚₑ model was applied to predict the nAb binding strength (K_{d}) for each of the LHS-sample. FIG. 15 shows the heatmap of the epitope profiles for the 10⁵ samples (sorted by the total K_{d} scores of 4 nAbs) and their predicted nAb binding values (K_{d}). The heatmap results clearly show the optimal nAb affinity (the lowest total K_{d} scores) involves highly abundant exposed galactose epitopes (STL and RCA120) and is depleted in epitopes of bi-antennary exposed GlcNAc (PHA_E) and sialylated epitope (MAH). Indeed, the bar plots (FIG. 15) showed the same epitope abundance patterns for the top 5 NN-geCHO samples, which all lacked epitopes bound by PHA_E and MAH. Meanwhile, all had nearly maximum expression of epitopes bound by STL and RCA120. These 5 in-silico screened geCHOs were predicted to have excellent nAb binding efficacious on all the four interested nAbs (CBH-4D, AR3A, HC84.26, and HCV1) (Table 8). Specifically, the 1^{st} ranked NN-geCHO showed 15.5× and 6.4× improved binding to neutralizing antibodies compared to the glycoforms produced in CHO-S and HEK 293 cells (Tables 9 and 10), which is a two-fold improvement of the best geCHO tested (8.0× and 3.3× improvement respectively). Furthermore, since the 10⁵ different glycoforms have been comprehensively sampled (FIG. 16), the analysis thus also provides hints on the limit of the theoretical maximum nAb binding could be reached by the isolated nAbs and the geCHO glycoform. All the information can be useful for future rational glycoengineering to the host cells to produce subunit vaccines against HCV infection.

TABLE 8
Predicted nAb binding K_{d} of the top5 in-silico geCHO
clones using the best NN_{cpitopc} model

| | | | | | Total K_{d} |
|---|---|---|---|---|---|
| | CBH.4D | AR3A | HC84.26 | HCV1 | Score |
| WT(CHO) | 0.99 | 2.86 | 1.22 | 0.69 | 5.76 |
| WT(HEK) | 0.22 | 1.47 | 0.36 | 0.34 | 2.39 |
| geCHO#1 (best) | 0.08 | 0.18 | 0.06 | 0.4 | 0.72 |
| NN-geCHO (top1) | 0.03 | 0.12 | 0.05 | 0.17 | 0.37 |
| NN-geCHO (top2) | 0.03 | 0.14 | 0.03 | 0.17 | 0.37 |
| NN-geCHO (top3) | 0.02 | 0.16 | 0.04 | 0.16 | 0.38 |
| NN-geCHO (top4) | 0.03 | 0.17 | 0.04 | 0.18 | 0.42 |
| NN-geCHO (top5) | 0.03 | 0.14 | 0.04 | 0.20 | 0.42 |

TABLE 9
Fold change of nAb binding K_{d} of the selected
geCHO clones relative to WT(CHO)

| | | | | | Total K_{d} |
|---|---|---|---|---|---|
| | CBH.4D | AR3A | HC84.26 | HCV1 | Score |
| WT(CHO) | 1.0 | 1.0 | 1.0 | 1.0 | 1.0 |
| WT(HEK) | 4.5 | 1.9 | 3.4 | 2.0 | 2.4 |
| geCHO#1 (best) | 12.4 | 15.9 | 20.3 | 1.7 | 8.0 |
| NN-geCHO (top1) | 33.1 | 23.4 | 25.6 | 4.0 | 15.5 |
| NN-geCHO (top2) | 35.3 | 19.7 | 35.4 | 4.1 | 15.4 |
| NN-geCHO (top3) | 45.6 | 17.7 | 27.1 | 4.4 | 15.0 |
| NN-geCHO (top4) | 29.2 | 16.8 | 34.6 | 3.9 | 13.8 |
| NN-geCHO (top5) | 30.4 | 20.3 | 27.2 | 3.4 | 13.8 |

TABLE 10
Fold change of nAb binding K_{d} of the selected
geCHO clones relative to WT(HEK)

| | | | | | Total K_{d} |
|---|---|---|---|---|---|
| | CBH.4D | AR3A | HC84.26 | HCV1 | Score |
| WT(CHO) | 0.2 | 0.5 | 0.3 | 0.5 | 0.4 |
| WT(HEK) | 1.0 | 1.0 | 1.0 | 1.0 | 1.0 |
| geCHO#1 (best) | 2.8 | 8.2 | 6.0 | 0.9 | 3.3 |
| NN-geCHO | 7.3 | 12.0 | 7.6 | 2.0 | 6.4 |
| (top1) | | | | | |
| NN-geCHO | 7.8 | 10.1 | 10.4 | 2.0 | 6.4 |
| (top2) | | | | | |
| NN-geCHO | 10.1 | 9.1 | 8.0 | 2.2 | 6.2 |
| (top3) | | | | | |
| NN-geCHO | 6.5 | 8.6 | 10.2 | 1.9 | 5.7 |
| (top4) | | | | | |
| NN-geCHO | 6.7 | 10.4 | 8.0 | 1.7 | 5.7 |
| (top5) | | | | | |

2. Conclusions

Glycoengineering offers an opportunity to develop subunit vaccines that better approximate the native glycosylation of a virus for enhanced vaccine production. Furthermore, it can indicate further improvements to make nAb binding sites more accessible. Finally, it also provides further insights into how glycosylation influences variations in vaccine efficacy. Here the expected native glycoform of the HCV E2 protein was demonstrated, and hypothesized that approximating this native liver glycosylation would enhance the affinity of patient-derived nAbs to a candidate E2 subunit vaccine. A panel of E2 glycoforms were then produced and tested. The best glycoform showed 8× and 3.3× improved binding to neutralizing antibodies, compared to the glycoform produced in the wildtype CHO-S and HEK 293 cells. Importantly, the best candidate glycoforms all were far more similar to glycans seen on liver-derived proteins, with the best candidates also having reduced sialylation and branching, which likely increased nAb binding site accessibility with minimal changes to protein structure.

A machine learning-based glycoinformatic system was developed, the GlycoEpitope Positioning System (GEPS), to identify the specific glycoform and associated glycan epitopes resulting in the most effective subunit vaccine candidate. Moreover, an in-silico clone screening tool was developed for aid in identifying host cells for producing the optimal glycoform. The best in-silico screened glycoform showing 15.5× and 6.4× improved binding to neutralizing antibodies compared to the glycoform produced in the wildtype CHO-S and HEK 293 cells. The GEPS system presents a useful method to design a glycoprotein vaccine and provides a strategy for predicting candidate vaccine efficacy.

3. Materials and Methods

i. Protein Expression and Purification in HEK 293 and CHO Systems

For mammalian cell expression, a gene encoding HCV E2 from strain 1a H77c (residues 384 to 661) was cloned into the vector pSecTag2 (Invitrogen) with an N-terminal immunoglobulin a light-chain signal sequence (for secretion) and a C-terminal His6 tag (for purification). The construct was transfected with ExpiFectamine 293 into Expi293F cells (Invitrogen). Recombinant monomeric sE2.HEK was purified from culture supernatants by sequential HisTrap Ni²⁺-NTA and Superdex 200 columns (GE Healthcare). The same expression vector was used for CHO and geCHO expression (see transfection and expression details in FIG. 7B). The purification protocol used above was also used to isolate monomeric sE2.CHO and sE2.geCHO from culture supernatants.

ii. Enzyme-Linked Immunosorbent Assay (ELISA)

HCV HMAb binding to sE2.HEK, sE2.CHO and sE2.geCHO (FIGS. 1 and 2) were evaluated and quantified by ELISA. 96-well microplates (MaxiSorp, Thermo Fisher, Waltham, MA) were coated with 5 μg/mL Galanthus Nivalis Lectin (Vector Laboratories, Burlingame, CA) overnight, and purified sE2.HEK, sE2.CHO and sE2.geCHO was then added to the plates at 2 ug/ml. After the plates were washed with PBS and 0.05% Tween 20, and blocked by Pierce™ Protein-Free (PBS) Blocking Buffer (Thermo Fisher, Waltham, MA), the nAbs were tested in duplicate at 3-fold serial dilutions starting at 3 ug/ml. The binding was detected by 1:5000 dilutions of HRP-conjugated anti-human IgG secondary antibody (Invitrogen, Carlsbad, CA) with TMB substrate (Bio-Rad Laboratories, Hercules, CA). The absorbance was read at 450 nm using a SpectraMax MS microplate reader (Molecular Devices, San Jose, CA). Approximate Kds were calculated by non-linear regression in GraphPad Prism software. The results of these studies are presented in Table 2. Of the geCHO glycoforms examined, antigens containing glycoforms #1, 2, 4, and 7 exhibited tighter binding to antibodies directed against domains B and D than antigens produced in wild-type CHO or HEK 293 cells. In addition, antigens containing glycoforms #1 and 2 exhibited slightly tighter binding to an antibody directed against domain E. Moreover, antigens containing glycans #1, 2, 4, and 7 facilitate greater exposure of domain B to the antibody AR3A as evidenced by the increase in Bmax relative to antigens produced in both wild-type CHO and HEK cells.

iii. Annotating Glycomics Data with Glycan Structure & Glycan Epitopes Information

To glycoprofile recombinant sE2 protein, 12 μL of concentrated protein solutions (concentrations varying between 0.1 and 1 mg/mL) were subjected to N-glycan labeling using the GlycoWorks RapiFluor-MS N-Glycan Kit (Waters). Labeled N-glycans were analyzed by LC-MS as described previously⁴⁸. Initial conditions were 25% 50 mM ammonium formate buffer, 75% Acetonitrile, separation gradient from 30% to 43% buffer. Mass spectrometry was run in positive mode with no source fragmentation. The normalized, relative amount of the N-glycans is calculated from the area under the peak with Thermo Xcalibur software (Thermo Fisher Scientific).

To distinguish heterogeneous glycan antigens among the glycoprofiles, a set of glycan substructures (epitopes) need to be selected that can capture the entire glycome upon a broad-spectrum N-linked protein glycosylation. Thirteen lectins (Table 3) were manually selected that distinguish 13 specific glycan structural features of N-linked glycans.⁴⁹⁻⁵¹ Specifically, glycan structures were distinguished such as: the branches of N-linked glycans with a maximum of four branches (GlcNAc-β1,2/4/6), LacNAc elongation (GlcNAc-β1,3), epitope monosaccharides (e.g., fucose), and high mannose structures. Given a glycoprofile, the epitope profiles for given glycans were first generated by EPg_{i,j}=Glycanᵢ*W_{i,j}, where EPg_{i,j} is the epitope profiles for given glycans, in which each row represents a glycan and each column represents a glycan epitope; Glycanᵢ means glycan i of a known structure; and, W_{i,j} is the frequency of glycan motifs on glycan i recognized by lectin j; if glycan i cannot be recognized by lectin j, the value is 0. It should be noted that realistic W_{i,j} needs to be adjusted and will depend on the real binding affinities of chosen glycans to the expected epitopes. In this study, the calculation of the epitope profiles was simplified by ignoring the kinetics of lectin binding (given that binding will often be done to a steady state level), and the binding specificities of certain lectins will require further experimental validation. Then, the epitope profiles were generated by EP_{k,j}=GPg_{k,i}*EPg_{i,j}, where EP_{k,j} is the epitope profiles for given glycoprofiles, where each row represents a specific glycoprofile and each column represents a epitope; and, GPg_{k,i} is the signal intensity (relative MS/HPLC intensity) of glycan i in the given glycoprofile k. Here this method was applied to generate epitope profiles from the glycoprofiles of geCHOs (FIGS. 6 and 17-23) of 7 different glycoengineered CHO cell lines (#1,2,4,5,6,7,10). The resulting epitope profiles were used for further analysis in this study.

iv. Pair-Wise Correlation Analysis Between the Glycans/Epitopes and nAb Binding Strength

To determine pairwise correlation relationships between glycans (or epitopes) and nAb binding strength, the Spearman's rank correlation coefficient was used^{52,53}, which describes the directionality and strength of the relationship between two studied variables. The Spearman's rank correlation is a nonparametric measure using the rank values of the two variables, in which the more similar the expression profiles for all samples are, the higher the correlation coefficient will be. Specifically, pair-wise Spearman's rank correlation coefficients were calculated for the glycan-nAb and epitope-nAb, and the correlation results were visualized by using the corrplot package⁵⁴ in the R language.

v. Multinomial Linear Regression Model Based on the Glycan Epitope Profile

For modeling the relationships between multiple epitopes, the multiple regression analysis was applied⁴², wherein models take into account the correlations between several predictor epitopes and assess the effect of each predictor epitope on the response nAb binding strength (K_{d}). Compared with the pairwise correlation analysis that only studies one variable, the use of several variables improved the accuracy of predictions. Here the lm( ) function (‘stats’ package in the R language) was used to fit the linear models. To protect against overfitting, leave-one-out cross validation was applied, in which the learning process is applied once for each geCHO sample, using all other geCHO samples as a training set and using the selected geCHO sample as a single-item test set.^{43,44} Moreover, the best performing models were further subjected to ‘Akaike Information Criterion (AIC)’ analysis^{44,45} to select the most generalized predictor variables to avoid overfitting. Specifically, the AIC score was calculated by using the AIC( ) function (‘stats’ package in the R language) for the best leave-one-out trained models. The resulting models are presented in Tables 4-7 for further analyses.

vi. Neural Network Model Based on the Glycan/Epitope Profile

Neural networks were built to learn the relationship between glycan profiles (or epitope profiles) to the tested nAb binding (K_{d}) from the training data. Specifically, the glycoprofiles and the transformed epitope profiles for each glycoprofile were used. A neural network model was built to predict the nAb binding (K_{d}) profile from the glycan profiles (or epitope profiles). The ‘neuralnet’ package of R language was used to train the neural network model. A neural network consists of one or more hidden layers, each of which includes a number of neurons. The output of the neural network is the nAb binding (K_{d}) values. To protect against overfitting, the leave-one-out cross validation was applied, wherein the learning process is applied once for each geCHO sample, using all other geCHO samples as a training set and using the selected geCHO sample as a single-item test set.^{43,44} To determine the optimal neural network topology, different combinations of hidden layer size (1 to 5 layers) and neuron size (10-50 neurons) in each layer were analyzed (FIG. 12). The best performed neural networks were selected for variable importance analysis and sensitivity analysis of predictor variables. Specifically, the importance of predictor variables were further calculated using olden(function^{45,46} (‘NeuralNetTools’ package) of the R language. The sensitivity analysis was performed using code developed in-house by modifying the functions used in the ‘NeuralNetTools’ package⁵⁶ of the R language.

### B. Example 2 Using Glycoengineered Proteins as Probes for Novel Antibodies

The COVID-19 pandemic caused by the SARS-CoV-2 virus has made the development of safe and effective vaccines and therapeutics a global priority. The rapidly accumulating data on protective SARS-CoV-2 immune responses, vaccine delivery approaches, and structures that describe the spike glycoprotein and its engagement by neutralizing antibodies provide the opportunity to generate both optimized vaccines and therapeutics. The primary target of vaccines and therapeutic antibodies in use and development is the SARS-CoV-2 spike glycoprotein. Spike is a heavily glycosylated trimer, with 66 glycans per trimer. It has been established in general and specifically for SARS CoV-2 spike that glycans can impact immune recognition and thereby will affect strategies for developing effective rationally designed vaccines and therapeutic antibodies. In many cases, glycans on the glycoprotein surface interact directly with a neutralizing antibody, forming a part of the epitope, indicating that specific glycoforms and glycan profiles would be an important factor in isolating virus-specific and protective antibodies. The spike glycoprotein is presented on the surface of the SARS CoV-2 virus containing glycans conferred by the host cell (i.e., primary lung epithelial cells). Recognition by the immune system will thus be biased towards spike proteins containing glycans that resemble those present in a natural infection. Recombinant spike proteins are produced using CHO cells engineered to confer glycans of the same structure and content as the most abundant glycans conferred by lung epithelial cells.

To produce the spike trimer, a construct has been synthesized that mimics a previously reported expression system used for structural studies. This construct replicates the 2P variant used to stabilize the spike ectodomains of SARS CoV and MERS CoV in the prefusion conformation. Trimer formation results from inclusion of the T4 fibritin trimerization motif and both His8 and twin strep tags are included to facilitate purification. Our purification regimen includes immobilized metal affinity chromatography (IMAC) followed by polishing by size exclusion chromatography (SEC) to yield highly-purified material. Using this expression system, we will produce the spike trimer in eight different CHO cell lines (wild-type and seven glycoengineered lines). The seven glycoengineered cell lines confer glycoforms likely to be present in primary lung epithelial cells. These lines are shown in Table 11.

TABLE 11
CHO cell lines used for spike production

| Cell line | Identification Number | Glycan features |
|---|---|---|
| 1 | CHO_S (wild-type) | WT |
| 2 | CHO_S_176_2(43-1#1-E1) | Triantennary Fucosylated |
| 3 | CHO_S_203_4(45-1#2-C12) | Biantennary fucosylated |
| 4 | CHO_S_203_2(38-1#1-C7) | Biantennary a-fucosylated |
| 5 | CHO_S_47_2_6_5(3-3-1#E1) | Biantennary human sialylated |
| 6 | CHO_S_47_2_7(45-1#3-A10) | Biantennary a-sialylated |
| 7 | CHO_S_45_1_9(40-1#3-B12) | Monoantennary glycans |
| 8 | CHO_S_32_2_2(49-1#1-B6) | Minimum monoantennary |

These probes can then be used to screen for broadly neutralizing antibodies in patient plasma using methods previously employed to isolate neutralizing antibodies directed against HIV gp120 (Sajadi M M, et al. Cell. 2018; 173(7):1783-95). Briefly, the fraction containing broadly neutralizing antibodies can be enriched via affinity chromatography and free flow isoelectric focusing. This enriched fraction can be subjected to enzymatic digestion and the resulting peptide fragments analyzed by liquid chromatography-tandem mass spectrometry (LC-MS/MS) to calculate mass assignments, and thereby sequences of the individual peptides. These peptide sequences can enable identification of the Ig H and L chain genes from which they originated.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Grubaugh, N. D. et al. Tracking virus outbreaks in the twenty-first century. Nature Microbiology (2019) doi:10.1038/s41564-018-0296-2.
2. Stanaway, J. D. et al. The global burden of viral hepatitis from 1990 to 2013: findings from the Global Burden of Disease Study 2013. Lancet (2016) doi:10.1016/S0140-6736(16)30579-7.
3. El-Serag, H. B. Epidemiology of viral hepatitis and hepatocellular carcinoma. Gastroenterology (2012) doi:10.1053/j.gastro.2011.12.061.
4. Shepard, C. W., Finelli, L. & Alter, M. J. Global epidemiology of hepatitis C virus infection. Lancet Infectious Diseases (2005) doi:10.1016/S1473-3099(05)70216-4.
5. Mysore, K. R. & Leung, D. H. Hepatitis B and C. Clinics in Liver Disease (2018) doi:10.1016/j.cld.2018.06.002.
6. Asrani, S. K., Devarbhavi, H., Eaton, J. & Kamath, P. S. Burden of liver diseases in the world. Journal of Hepatology (2019) doi:10.1016/j.jhep.2018.09.014.
7. Graham, B. S. & Sullivan, N. J. Emerging viral diseases from a vaccinology perspective: Preparing for the next pandemic review-article. Nature Immunology (2018) doi:10.1038/s41590-017-0007-9.
8. McAleer, W. J. et al. Human hepatitis B vaccine from recombinant yeast. Nature (1984) doi:10.1038/307178a0.
9. Poland, G. A. & Jacobson, R. M. Prevention of hepatitis B with the hepatitis B vaccine. New England Journal of Medicine (2004) doi:10.1056/NEJMcp041507.
10. Ogholikhan, S. & Schwarz, K. B. Hepatitis vaccines. Vaccines (2016) doi:10.3390/vaccines4010006.
11. Liang, T. J. Current progress in development of hepatitis C virus vaccines. Nature Medicine (2013) doi:10.1038/nm.3183.
12. Houghton, M. & Abrignani, S. Prospects for a vaccine against the hepatitis C virus. Nature (2005) doi:10.1038/nature04081.
13. Law, M. et al. Broadly neutralizing antibodies protect against hepatitis C virus quasispecies challenge. Nat. Med. (2008) doi:10.1038/nm1698.
14. Manns, M. P. et al. Hepatitis C virus infection. Nat. Rev. Dis. Prim. (2017) doi:10.1038/nrdp.2017.6.
15. Lauer, G. M. & Walker, B. D. Hepatitis C virus infection. New England Journal of Medicine (2001) doi:10.1056/NEJM200107053450107.
16. Mehta, A., Herrera, H. & Block, T. Glycosylation and liver cancer. Adv. Cancer Res. 126, 257-279 (2015).
17. Kong, L. et al. Hepatitis C virus E2 envelope glycoprotein core structure. Science (80-.). (2013) doi:10.1 126/science.1243876.
18. Khan, A. G. et al. Structure of the core ectodomain of the hepatitis C virus envelope glycoprotein 2. Nature (2014) doi:10.1038/nature13117.
19. Chiang, A. W. T., Wu, W. Y. L., Wang, T. & Hwang, M. J. Identification of Entry Factors Involved in Hepatitis C Virus Infection Based on Host-Mimicking Short Linear Motifs. PLoS Comput. Biol. 13, 1-24 (2017).
20. Goffard, A. et al. Role of N-Linked Glycans in the Functions of Hepatitis C Virus Envelope Glycoproteins. J. Virol. (2005) doi:10.1 128/jvi.79.13.8400-8409.2005.
21. Ghasemi, F., Rostami, S. & Meshkat, Z. Progress in the development of vaccines for hepatitis C virus infection. World J. Gastroenterol. (2015) doi:10.3748/wjg.v21.i42.11984.
22. Ghasemi, F., Ghayour-Mobarhan, M., Gouklani, H. & Meshkat, Z. Development of preventive vaccines for hepatitis C virus E1/E2 protein. Iranian Journal of Pathology (2018) doi:10.30699/ijp.13.2.113.
23. Dalziel, M., Crispin, M., Scanlan, C. N., Zitzmann, N. & Dwek, R. A. Emerging principles for the therapeutic exploitation of glycosylation. Science (80-.). 343, (2014).
24. Astronomo, R. D. & Burton, D. R. Carbohydrate vaccines: developing sweet solutions to sticky situations? Nat. Rev. Drug Discov. 9, 308-324 (2010).
25. Roy, R. New trends in carbohydrate-based vaccines. Drug Discov. Today Technol. 1, 327-336 (2004).
26. Wua, C. Y. et al. Influenza A surface glycosylation and vaccine design. Proc. Natl. Acad. Sci. U.S.A (2017) doi:10.1073/pnas.1617174114.
27. Hariharan, V. & Kane, R. S. Glycosylation as a tool for rational vaccine design. Biotechnology and Bioengineering (2020) doi:10.1002/bit.27361.
28. Horiya, S., MacPherson, I. S. & Krauss, I. J. Recent strategies targeting HIV glycans in vaccine design. Nature chemical biology (2014) doi:10.1038/nchembio.1685.
29. Wilson, J. T. A sweeter approach to vaccine design. Science (2019) doi:10.1126/science.aav9000.
30. Galili, U. Amplifying immunogenicity of prospective Covid-19 vaccines by glycoengineering the coronavirus glycan-shield to present α-gal epitopes. Vaccine (2020) doi:10.1016/j.vaccine.2020.08.032.
31. Hyakumura, M. et al. Modification of Asparagine-Linked Glycan Density for the Design of Hepatitis B Virus Virus-Like Particles with Enhanced Immunogenicity. J. Virol. (2015) doi:10.1128/jvi.01123-15.
32. Joe, C. C. D. et al. Glycoengineered hepatitis B virus-like particles with enhanced immunogenicity. Vaccine (2020) doi:10.1016/j.vaccine.2020.03.007.
33. Urbanowicz, R. A. et al. Antigenicity and Immunogenicity of Differentially Glycosylated Hepatitis C Virus E2 Envelope Proteins Expressed in Mammalian and Insect Cells. J. Virol. (2019) doi:10.1 128/jvi.01403-18.
34. Clerc, F. et al. Human plasma protein N-glycosylation. Glycoconj. J. (2016) doi:10.1007/s10719-015-9626-2.
35. Amann, T. et al. Glyco-engineered CHO cell lines producing alpha-1-antitrypsin and C1 esterase inhibitor with fully humanized N-glycosylation profiles. Metab. Eng. (2019) doi:10.1016/j.ymben.2018.11.014.
36. Zuber, V. & Strimmer, K. High-dimensional regression and variable selection using CAR scores. Statistical Applications in Genetics and Molecular Biology (2011) doi:10.2202/1544-6115.1730.
37. Stein, M. Large sample properties of simulations using latin hypercube sampling. Technometrics (1987) doi:10.1080/00401706.1987.10488205.
38. Li, D. et al. Altered Glycosylation Patterns Increase Immunogenicity of a Subunit Hepatitis C Virus Vaccine, Inducing Neutralizing Antibodies Which Confer Protection in Mice. J. Virol. (2016) doi:10.1 128/jvi.01462-16.
39. Revie, D. & Salahuddin, S. Z. Human cell types important for Hepatitis C Virus replication in vivo and in vitro. Old assertions and current evidence. Virology Journal (2011) doi:10.1 186/1743-422X-8-346.
40. Bekesova, S. et al. N-glycans in liver-secreted and immunoglogulin-derived protein fractions. J. Proteomics (2012) doi:10.1016/j.jprot.2012.01.024.
41. Eberly, L. E. Multiple linear regression. Methods in molecular biology (Clifton, N.J.) (2007) doi:10.1007/978-1-59745-530-5_9.
42. Bremer, M. Multiple Linear Regression. Handb. Regres. Anal. 1-21 (2013) doi:10.1002/9781118532843.chl.
43. Webb, G. I. et al. Leave-One-Out Cross-Validation. in Encyclopedia of Machine Learning (2011). doi:10.1007/978-0-387-30164-8_469.
44. Shao, J. Linear model selection by cross-validation. J. Am. Stat. Assoc. (1993) doi:10.1080/01621459.1993.10476299.
45. Olden, J. D., Joy, M. K. & Death, R. G. An accurate comparison of methods for quantifying variable importance in artificial neural networks using simulated data. Ecol. Modell. (2004) doi:10.1016/j.ecolmodel.2004.03.013.
46. Olden, J. D. & Jackson, D. A. Illuminating the “black box”: A randomization approach for understanding variable contributions in artificial neural networks. Ecol. Modell. (2002) doi:10.1016/S0304-3800(02)00064-9.
47. Garson, D. Interpreting neural-network connection strengths. AI Expert (1991).
48. Amann, T., Schmieder, V., Faustrup Kildegaard, H., Borth, N. & Andersen, M. R. Genetic engineering approaches to improve posttranslational modification of biopharmaceuticals in different production platforms. Biotechnology and Bioengineering (2019) doi:10.1002/bit.27101.
49. Tateno, H. et al. A novel strategy for mammalian cell surface glycome profiling using lectin microarray. Glycobiology (2007) doi:10.1093/glycob/cwm084.
50. Malik, A., Lee, J. & Lee, J. Community-based network study of protein-carbohydrate interactions in plant lectins using glycan array data. PLoS One (2014) doi:10.1371/journal.pone.0095480.
51. Michiels, K., Van Damme, E. J. & Smagghe, G. Plant-insect interactions: What can we learn from plant lectins? Arch. Insect Biochem. Physiol. (2010) doi:10.1002/arch.20351.
52. Fieller, E. C., Hartley, H. O. & Pearson, E. S. Tests for Rank Correlation Coefficients. I. Biometrika (1957) doi:10.2307/2332878.
53. Rider, P. R. On the Distribution of the Correlation Coefficient in Small Samples. Biometrika (1932) doi:10.2307/2331973.
54. Friendly, M. Corrgrams: Exploratory displays for correlatigon matrices. Am. Stat. (2002) doi:10.1198/000313002533.
55. Akaike information criterion statistics. Math. Comput. Simul. (1987) doi:10.1016/0378-4754(87)90094-2.
56. Beck, M. W. NeuralNetTools: Visualization and analysis tools for neural networks. J. Stat. Softw. (2018) doi:10.18637/jss.v085.i11.
Watanabe Y, Allen J D, Wrapp D, McLellan J S, Crispin M. Site-specific analysis of the SARS-CoV-2 glycan shield. bioRxiv. 2020. doi: 10.1101/2020.03.26.010322. PubMed PMID: 32511336; PMCID: PMC7239077.
Urbanowicz R A, Wang R, Schiel J E, Keck Z Y, Kerzic M C, Lau P, Rangarajan S, Garagusi K J, Tan L, Guest J D, Ball J K, Pierce B G, Mariuzza R A, Foung S K H, Fuerst T R. Antigenicity and Immunogenicity of Differentially Glycosylated Hepatitis C Virus E2 Envelope Proteins Expressed in Mammalian and Insect Cells. J Virol. 2019; 93(7). Epub 2019/01/18. doi: 10.1128/JVI.01403-18. PubMed PMID: 30651366; PMCID: PMC6430559.
Helle F, Goffard A, Morel V, Duverlie G, McKeating J, Keck Z Y, Foung S, Penin F, Dubuisson J, Voisset C. The neutralizing activity of anti-hepatitis C virus antibodies is modulated by specific glycans on the E2 envelope protein. J Virol. 2007; 81(15):8101-11. doi: 10.1128/JVI.00127-07. PubMed PMID: 17522218; PMCID: 1951279.
Zhou T, Doria-Rose N A, Cheng C, Stewart-Jones G B E, Chuang G Y, Chambers M, Druz A, Geng H, McKee K, Kwon Y D, O'Dell S, Sastry M, Schmidt S D, Xu K, Chen L, Chen R E, Louder M K, Pancera M, Wanninger T G, Zhang B, Zheng A, Farney S K, Foulds K E, Georgiev I S, Joyce M G, Lemmin T, Narpala S, Rawi R, Soto C, Todd J P, Shen C H, Tsybovsky Y, Yang Y, Zhao P, Haynes B F, Stamatatos L, Tiemeyer M, Wells L, Scorpio D G, Shapiro L, McDermott A B, Mascola J R, Kwong P D. Quantification of the Impact of the HIV-1-Glycan Shield on Antibody Elicitation. Cell Rep. 2017; 19(4):719-32. Epub 2017/04/27. doi: 10.1016/j.celrep.2017.04.013. PubMed PMID: 28445724; PMCID: PMC5538809.
Medina R A, Stertz S, Manicassamy B, Zimmermann P, Sun X, Albrecht R A, Uusi-Kerttula H, Zagordi O, Belshe R B, Frey S E, Tumpey T M, Garcia-Sastre A. Glycosylations in the globular head of the hemagglutinin protein modulate the virulence and antigenic properties of the H1N1 influenza viruses. Sci Transl Med. 2013; 5(187):187ra70. doi: 10.1 126/scitranslmed.3005996. PubMed PMID: 23720581; PMCID: PMC3940933.
Berndsen Z T, Chakraborty S, Wang X, Cottrell C A, Torres J L, Diedrich J K, Lopez C A, Yates J R, 3rd, van Gils M J, Paulson J C, Gnanakaran S, Ward A B. Visualization of the HIV-1 Env glycan shield across scales. Proc Natl Acad Sci USA. 2020; 117(45):28014-25. Epub 2020/10/24. doi: 10.1073/pnas.20002601 17. PubMed PMID: 33093196; PMCID: PMC7668054.
Grant O C, Montgomery D, Ito K, Woods R J. Analysis of the SARS-CoV-2 spike protein glycan shield reveals implications for immune recognition. Scientific reports. 2020; 10(1):14991. Epub 2020/09/16. doi: 10.1038/s41598-020-71748-7. PubMed PMID: 32929138; PMCID: PMC7490396.
Acharya P, Williams W, Henderson R, Janowska K, Manne K, Parks R, Deyton M, Sprenz J, Stalls V, Kopp M, Mansouri K, Edwards R J, Meyerhoff R R, Oguin T, Sempowski G, Saunders K, Haynes B F. A glycan cluster on the SARS-CoV-2 spike ectodomain is recognized by Fab-dimerized glycan-reactive antibodies. bioRxiv. 2020. Epub 2020/07/09. doi: 10.1101/2020.06.30.178897. PubMed PMID: 32637953; PMCID: PMC7337383.
Kong L, Giang E, Nieusma T, Kadam R U, Cogburn K E, Hua Y, Dai X, Stanfield R L, Burton D R, Ward A B, Wilson I A, Law M. Hepatitis C virus E2 envelope glycoprotein core structure. Science. 2013; 342(6162):1090-4. doi: 10.1126/science.1243876. PubMed PMID: 24288331; PMCID: 3954638.
Pancera M, Shahzad-Ul-Hussan S, Doria-Rose N A, McLellan J S, Bailer R T, Dai K, Loesgen S, Louder M K, Staupe R P, Yang Y, Zhang B, Parks R, Eudailey J, Lloyd K E, Blinn J, Alam S M, Haynes B F, Amin M N, Wang L X, Burton D R, Koff W C, Nabel G J, Mascola J R, Bewley C A, Kwong P D. Structural basis for diverse N-glycan recognition by HIV-1-neutralizing V1-V2-directed antibody PG16. Nat Struct Mol Biol. 2013; 20(7):804-13. Epub 2013/05/28. doi: 10.1038/nsmb.2600. PubMed PMID: 23708607; PMCID: PMC4046252.
Julien J P, Sok D, Khayat R, Lee J H, Doores K J, Walker L M, Ramos A, Diwanji D C, Pejchal R, Cupo A, Katpally U, Depetris R S, Stanfield R L, McBride R, Marozsan A J, Paulson J C, Sanders R W, Moore J P, Burton D R, Poignard P, Ward A B, Wilson I A. Broadly neutralizing antibody PGT121 allosterically modulates CD4 binding via recognition of the HIV-1 gp120 V3 base and multiple surrounding glycans. PLoS Pathog. 2013; 9(5):e1003342. Epub 2013/05/10. doi: 10.1371/journal.ppat.1003342. PubMed PMID: 23658524; PMCID: PMC3642082.
Barnes C O, Gristick H B, Freund N T, Escolano A, Lyubimov A Y, Hartweger H, West A P, Jr., Cohen A E, Nussenzweig M C, Bjorkman P J. Structural characterization of a highly-potent V3-glycan broadly neutralizing antibody bound to natively-glycosylated HIV-1 envelope. Nat Commun. 2018; 9(1):1251. Epub 2018/03/30. doi: 10.1038/s41467-018-03632-y. PubMed PMID: 29593217; PMCID: PMC5871869.
Calarese D A, Scanlan C N, Zwick M B, Deechongkit S, Mimura Y, Kunert R, Zhu P, Wormald M R, Stanfield R L, Roux K H, Kelly J W, Rudd P M, Dwek R A, Katinger H, Burton D R, Wilson I A. Antibody domain exchange is an immunological solution to carbohydrate cluster recognition. Science. 2003; 300(5628):2065-71. Epub 2003/06/28. doi: 10.1126/science.1083182. PubMed PMID: 12829775.
Wrapp D, Wang N, Corbett K S, Goldsmith J A, Hsieh C L, Abiona O, Graham B S, McLellan J S. Cryo-EM structure of the 2019-nCoV spike in the prefusion conformation. Science. 2020; 367(6483):1260-3. Epub 2020/02/23. doi: 10.1 126/science.abb2507. PubMed PMID: 32075877.
Pallesen J, Wang N, Corbett K S, Wrapp D, Kirchdoerfer R N, Turner H L, Cottrell C A, Becker M M, Wang L, Shi W, Kong W P, Andres E L, Kettenbach A N, Denison M R, Chappell J D, Graham B S, Ward A B, McLellan J S. Immunogenicity and structures of a rationally designed prefusion MERS-CoV spike antigen. Proc Natl Acad Sci USA. 2017; 114(35):E7348-E57. Epub 2017/08/16. doi: 10.1073/pnas.1707304114. PubMed PMID: 28807998; PMCID: PMC5584442.
Yang X, Lee J, Mahony E M, Kwong P D, Wyatt R, Sodroski J. Highly stable trimers formed by human immunodeficiency virus type 1 envelope glycoproteins fused with the trimeric motif of T4 bacteriophage fibritin. J Virol. 2002; 76(9):4634-42. doi: 10.1 128/jvi.76.9.4634-4642.2002. PubMed PMID: 11932429; PMCID: PMC155086.
Sajadi M M, Dashti A, Rikhtegaran Tehrani Z, Tolbert W D, Seaman M S, Ouyang X, Gohain N, Pazgier M, Kim D, Cavet G, Yared J, Redfield R R, Lewis G K, DeVico A L. Identification of Near-Pan-neutralizing Antibodies against HIV-1 by Deconvolution of Plasma Humoral Responses. Cell. 2018; 173(7):1783-95 e14. Epub 2018/05/08. doi: 10.1016/j.cell.2018.03.061. PubMed PMID: 29731169; PMCID: PMC6003858.

We claim:

1. A method of producing a glyco-modified viral antigen comprising: expressing a viral antigen in a recombinant mammalian cell line having:
a. one or more of the endogenous genes Mgat2, Mgat4A, Mgat4B, Mgat5, St3Gal3, St3Gal4, B4galt1, B4galt2, B4galt3, B4galt4, B4galt5, B3gnt2, St3Gal6, SPPL3, and/or FUT8 inactivated and/or downregulated; and
b. optionally a ST6Gall gene.

2. The method of claim 1, wherein the viral antigen is a viral antigen from an enveloped virus.

3. The method of claim 2, wherein the enveloped virus is a DNA virus, RNA virus or a retrovirus.

4. The method of claim 3, wherein the DNA virus is a Herpesvirus, Poxvirus, Hepadnavirus, or an Asfarviridae.

5. The method of claim 3, wherein the RNA virus is a Flavivirus, Alphavirus, Togavirus, Coronavirus, Hepatitis D, Orthomyxovirus, Paramyxovirus, Rhabdovirus, Bunyavirus, or a Filovirus.

6. The method of claim 1, wherein the viral antigen is a HCV E2 glycoprotein.

7. The method of claim 6, wherein the HCV E2 glycoprotein is a modified HCV E2 glycoprotein.

8. The method of claim 7, wherein the modified HCV E2 glycoprotein comprises an antigenic domain D, wherein the modified HCV E2 glycoprotein comprises one or more amino acid alterations in the antigenic domain D.

9. The method of claim 1, wherein the viral antigen is a HCV E1E2 glycoprotein or a modified HCV E1E2 glycoprotein.

10. The method of claim 9, wherein the HCV E1E2 glycoprotein is a membrane bound HCV E1E2 glycoprotein.

11. The method of claim 10, wherein the membrane bound HCV E1E2 glycoprotein is a modified membrane bound HCV E1E2 glycoprotein.

12. The method of claim 11, wherein the modified membrane bound HCV E1E2 glycoprotein comprises an HCV E1 glycoprotein and a modified HCV E2 glycoprotein, wherein the modified HCV E2 glycoprotein comprises an antigenic domain D, wherein the modified HCV E2 glycoproteins comprise one or more amino acid alterations in the antigenic domain D.

13. The method of claim 9, wherein the modified HCV E1E2 glycoprotein comprises:
a. a HCV E1 polypeptide,
b. a first scaffold element,
c. a HCV E2 polypeptide, and
d. a second scaffold element
wherein the HCV E1 polypeptide does not comprise a transmembrane domain, and wherein the HCV E2 polypeptide does not comprise a transmembrane domain.

14. The method of any of the preceding claims, wherein the glyco-modified viral antigen has increased antigenicity compared to the viral antigen.

15. The method any of the preceding claims, wherein the glyco-modified viral antigen comprises one or more of the glycan structures:
a. a primary n-glycan structure that is a fully sialylated bi-antennary structure without core fucosylation, such as with more than 80%, such as 82%, such as 84%, such as 86%, such as 88%, such as 90% of the glycoproteins of interest produced being in with a fully sialylated bi-antennary structure without core fucosylation;
b. a glycan structure according to the structure A2G2S2 with the following pictorial representations: 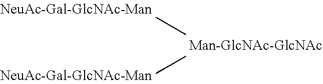
c. a glycan structure according to the structure: 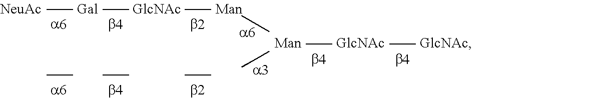
and
d. a glycan structure according to one or more of the structures: mono-antennary, no sialic acids (FA1G1); mono-antennary non-fucosylated, no sialic acids (A1G1); or mono-antennary non-fucosylated (FA1G1S1).

16. A glyco-modified viral antigen produced by the method of any of the preceding claims.

17. A glyco-modified hepatitis C virus (HCV) E2 glycoprotein comprising an antigenic domain D, wherein the modified hepatitis C virus (HCV) E2 glycoprotein comprises one or more amino acid alterations in the antigenic domain D and further comprising one or more of the glycan structures of Table 1.

18. The glyco-modified HCV E1E2 glycoprotein of claim 17, wherein the glycan structure is A2G2S2(6) Biantennary non-fucosylated α2,6 linked sialic acids.

19. A glyco-modified membrane bound hepatitis C virus (HCV) E1E2 glycoprotein comprising an HCV E1 glycoprotein and a modified HCV E2 glycoprotein, wherein the modified HCV E2 glycoprotein comprises an antigenic domain D, wherein the modified HCV E2 glycoproteins comprise one or more amino acid alterations in the antigenic domain D and further comprising one or more of the glycan structures of Table 1.

20. A glyco-modified hepatitis C virus (HCV) E1E2 glycoprotein comprising: a HCV E1 polypeptide, wherein the HCV E1 polypeptide does not comprise a transmembrane domain, a first scaffold element, a HCV E2 polypeptide, wherein the HCV E2 polypeptide does not comprise a transmembrane domain, and a second scaffold element and further comprising one or more of the glycan structures of Table 1.

21. The glyco-modified HCV E1E2 glycoprotein of claim 19 or 20, wherein the glycan structure is A2G2S2(6) Biantennary non-fucosylated α2,6 linked sialic acids.

22. A method of treating a subject in need thereof comprising: administering to the subject a composition comprising a therapeutically effective amount of one or more of the glyco-modified viral antigen of claims 16-21.

23. A method of increasing HCV E2 antigenicity in a subject in need thereof comprising: administering to the subject a composition comprising a glyco-modified HCV E2 produced using the method of any of claims 1-15 or one or more of the glyco-modified viral antigen of claims 17-21.

24. A method of inducing an immune response in a subject in need thereof comprising: administering to the subject a composition comprising a glyco-modified viral antigen produced using the method of any of claims 1-15 or one or more of the glyco-modified viral antigens of claims 16-21.

25. A method of treating a subject having HCV comprising administering to the subject a composition comprising a glyco-modified HCV E2 produced using the method of any of claims 1-15 or one or more of the glyco-modified viral antigen of claims 17-21.

26. A method of identifying an antibody specific to one or more of the glyco-modified viral antigen of claims 16-21 comprising: contacting a cell with one or more of the modified viral antigens of claims 16-21 under conditions to allow for the cell to elicit and immune response, isolating an antibody that specifically binds to the one or more of the modified viral antigens.

27. The method of claim 26, wherein the method is carried out in a subject.

28. A method of generating an antibody specific to one or more of the modified viral antigens of claims 16-21 comprising: contacting a cell with one or more of the modified viral antigens of claims 16-21 under conditions to allow for the cell to elicit an immune response, isolating an antibody that specifically binds to the one or more of the modified viral antigens.

29. The method of claim 28, wherein the method is carried out in a subject.

30. A method of screening for an antibody specific to one or more of the modified viral antigens of claims 16-21 comprising:
a. obtaining or having obtained an antibody from a subject;
b. contacting the antibody with one or more of the modified viral antigens of claims 16-21;
c. assessing binding strength (Kd) of the antibody to the one or more of the modified viral antigens; and
d. correlating the abundance of the one or more of the modified viral antigens and binding strength (Kd) of the antibody;
wherein an improved binding strength (Kd) relative to an antibody contacted with a non-glyco-modified viral antigen is an antibody specific to one or more of the disclosed glyco-modified viral antigens.

31. A method of screening for an antibody specific to one or more of the modified viral antigens of claims 16-21 comprising contacting a cell with one or more of the modified viral antigens of claims 16-21 under conditions to allow for the cell to elicit an immune response, identifying and isolating an antibody that specifically binds to the one or more of the modified viral antigens.

32. The method of claim 31, wherein the method is carried out in a subject.
